(19) United States
(12) Patent Application Publication
(10) Pub. No.: US 2006/0089647 A1
(43) Pub. Date: Apr. 27, 2006

(54) Method and apparatus for delivering an agent

(76) Inventors: Brad S. Culbert, Santa Margarita, CA (US); Christopher R. Warren, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/207,614

(22) Filed: Aug. 19, 2005

Related U.S. Application Data
(60) Provisional application No. 60/603,176, filed on Aug. 20, 2004.

(51) Int. Cl. A61F 2/30

(52) U.S. Cl. 606/65

(57) ABSTRACT

A fracture fixation device is configured for reducing and compressing fractures in a bone and delivering bone treatment agents. The fixation device includes an elongate body comprising a first portion and a second portion that are detachably coupled to each other. The first portion defines a helical cancellous bone anchor and the second portion defines a distal end. An axially moveable proximal anchor is carried by the proximal end of the fixation device and is rotationally locked to the first portion. The device is rotated into position across the femoral neck and into the femoral head, and the proximal anchor is distally advanced to lock the device into place. The second portion is then detached from the first portion.

### RELATED APPLICATIONS

This application claims the priority benefit under 35 U.S.C. § 119(e) of the provisional application 60/603,176, filed Aug. 20, 2004, which is hereby incorporated by reference in its entirety.

### BACKGROUND OF THE INVENTION

1. Field of the Invention

The present invention relates to bone fixation devices and medical agents. In one application, the present invention relates to bone fixation devices and methods adapted for delivery of a medical agent via the bone fixation devices.

2. Description of the Related Art

The femur, otherwise known as the thigh bone, generally comprises an elongate shaft extending from the hip to the knee. The proximal end of the shaft includes a head, a neck, a greater trochanter and a lesser trochanter. The head of the femur fits into the acetabular cup of the hip bone to form a ball and socket joint at the hip. The distal end of the femur includes a medial condyle and a lateral condyle. The condyles engage an upper end of the tibia to form the knee joint. Overall, the femur is the longest and strongest bone in the skeleton. However, portions of the femur are extremely susceptible to fracturing.

Pertrochanteric fractures among geriatric patients are the most frequent in connection with those of the region of the neck of the bone. The advanced age and the pathologies which are encountered in these patients make a timely stabilization of skeletal injuries necessary in order to reduce to a minimum the bed confinement and the rehabilitation times. Preferably, devices and procedures are utilized which minimize complications brought about by the so-called immobilization syndrome, which may be lethal for patients in delicate metabolical circumstances. It is also preferable to reduce to a minimum blood losses related to surgical intervention. At the same time, the syntheses means utilized must be stable in order to allow the patient to very timely assume a seated position and, two or three days following the intervention, to reassume an erect posture with progressive bearing of weight.

Internal fixation of femoral fractures in general is one of the most common orthopedic surgical procedures. Fractures of the femur occur in both the proximal portion of the femur and the distal portion of the femur. Fractures of the proximal portion of the femur (hip fractures) are generally classified as femoral neck fractures, intertrochanteric fractures and subtrochanteric fractures. Fractures of the distal portion of the femur (knee fractures) are referred to as supracondylar fractures. Supracondylar fractures generally extend vertically between the condyles at the lower end of the femur to separate the distal portion of the femur into two main bone fragments. A fracture line may be further comminuted to create a plurality of smaller bone fragments. Fractures of the femur which extend into the neck of the bone are generally more difficult to treat than fractures restricted to the shaft of the femur.

Operative treatment of the fractures requires that the fractures be internally fixed and possibly compressed. Fractures of the neck, head or trochanters of the femur have been treated with a variety of compression screw assemblies which include generally a compression plate having a barrel member, a lag screw and a compressing screw. The compression plate is secured to the exterior of the femur and the barrel member is inserted into a predrilled hole in the direction of the femoral head. The lag screw which has a threaded end and a smooth portion is inserted through the barrel member so that it extends across the break and into the femoral head. The threaded portion engages the femoral head. The compressing screw connects the lag screw to the plate. By adjusting the tension of the compressing screw the compression (reduction) of the fracture can be adjusted.

A variety of elongated implants (nail, screw, pin, etc.) have been developed, which are adapted to be positioned along the longitudinal axis of the femoral neck with a leading (distal) end portion in the femoral head so as to stabilize a fracture of the femoral neck. The elongated implant may be implanted by itself or connected to another implant such as a side plate or intramedullary rod. The leading end portion of the implant typically includes means to positively grip the femoral head bone (external threads, expanding arms, etc.), but the inclusion of such gripping means can introduce several significant problems. First, implants with sharp edges on the leading end portion, such as the externally threaded implants, exhibit a tendency to migrate proximally towards the hip joint weight bearing surface after implantation. This can occur when the proximal cortical bone has insufficient integrity to resist distal movement of the screw head. Such proximal migration under physiological loading, which is also referred to as femoral head cut-out, can lead to significant damage to the adjacent hip joint. Also, the externally threaded implants can generate large stress concentrations in the bone during implantation which can lead to stripping of the threads formed in the bone and thus a weakened grip. The movable arms of known expanding arm devices are usually free at one end and attached at the other end to the main body of the leading end portion of the implant. As a result, all fatigue loading is concentrated at the attached ends of the arms and undesirably large bending moments are realized at the points of attachment. In addition, conventional threaded implants generally exhibit insufficient holding power under tension, such that the threads can be stripped out of the femoral head either by overtightening during the implantation procedure or during post operative loading by the patient's weight.

Additionally, there are known devices and methods for delivering bone cement and growth factor to a bone. However, these devices and methods are unsatisfactory as they may involve complicated devices and methods of delivery.

Thus, notwithstanding the variety of efforts in the prior art, there remains a need for an orthopedic fixation device with improved locking force such as within the femoral head in a femoral neck application, which resists migration and rotation, and which can be used to deliver a medical agent such as bone cement or growth factor.

### SUMMARY OF THE INVENTION

Thus, in one embodiment, a method of securing a first bone fragment to a second bone fragment comprises providing a fixation device comprising an elongate body and a proximal anchor. The elongate body has a proximal end, a distal end with a distal anchor thereon and a lumen extending through the elongate body. The proximal anchor is carried by the elongate body and has complementary structures for permitting axial travel of the proximal anchor with respect to the elongate body in a distal direction but resisting axial travel of the proximal anchor with respect to the elongate body in a proximal direction. The method further comprises providing a delivery pin having a distal end, a proximal end and delivery lumen extending therethrough. The distal end of the delivery pin is configured to engage the proximal end of the elongate body. The fixation device is distally advanced to a first position with respect to the first and second bone fragments. A medical agent is delivered to the fixation device by injecting the medical agent through the delivery lumen of the delivery pin and into the lumen of the elongate body. The delivery pin is detached from the elongate body.

In another embodiment, a bone fixation system comprises a fixation device comprising an elongate body and a proximal anchor. The elongate body has a proximal end, a distal end with a distal anchor thereon and a lumen extending through the elongate body. The proximal anchor is carried by the elongate body and has complementary structures for permitting axial travel of the proximal anchor with respect to the elongate body in a distal direction but resisting axial travel of the proximal anchor with respect to the elongate body in a proximal direction. A delivery pin has a distal end, a proximal end and a delivery lumen extending therethrough. The distal end of the delivery pin is configured to be coupled to the fixation body to place the delivery lumen in communication with the first lumen. An elongated drive body has a proximal end and a distal end. A cannula extends through the elongated drive body and is configured to surround a portion of the delivery pin when the distal end of the elongated drive body is coupled to the proximal anchor of the bone fixation device. A handle is releasable coupled to the proximal end of the driver body, wherein removing the handle from the drive body exposes the proximal end of the delivery pin which extends through the drive body.

In another embodiment, a bone fixation system comprises a fixation device comprising an elongate body and a proximal anchor. The elongate body has a proximal end, a distal end with a distal anchor thereon and a lumen extending through the elongate body. The proximal anchor is carried by the elongate body and has complementary structures for permitting axial travel of the proximal anchor with respect to the elongate body in a distal direction but resisting axial travel of the proximal anchor with respect to the elongate body in a proximal direction. An elongated drive body has a proximal end, a distal end and a cannula extending therethrough. The distal end of the elongated drive body is configured to engage the proximal anchor such that rotation of the drive body causes rotation of the proximal anchor. The proximal end of the drive body includes a handle. A delivery pin has a distal end, a proximal end and a delivery lumen extending therethrough. The distal end of the delivery pin is configured to be coupled to the fixation body to place the delivery lumen in communication with the first lumen. The delivery pin extends through the cannular of the elongated body such that the distal end of the delivery pin may be coupled to the proximal end of the elongate body while the proximal end of the elongate body extends out through the handle.

In yet another embodiment, a method of securing a first bone fragment to a second bone fragment, comprising distally moving a fixation device to a first position. The fixation device comprising an elongate body, having a proximal end and a distal end, a helical anchor on the distal end of the elongate body and a proximal anchor moveably carried by the elongate body. The elongate body and the proximal anchor having complementary retention structures configured to resist proximal movement of the proximal anchor with respect to the elongate body. A first material is delivered through and out of the fixation device when the fixation device is located in the first position. The fixation device is moved from the first position to a second position, the second position being distal to the first position. A second material is delivered through and out of the fixation device when the fixation device is in the second position.

In another embodiment, a femoral neck fracture fixation device, comprising an elongate body, having a distal portion, a delivery pin and lumen extending therethrough. The distal portion includes a helical bone anchor. The delivery pin and distal portion are detachably coupled to each other at a junction. A proximal anchor is moveably carried by the body and comprises a tubular sleeve that in a first position extends distally past the junction between the distal portion and the delivery pin. Complementary retention structures are between the proximal anchor and the elongate body. The complementary retention structures are configured to restrain proximal movement of the proximal anchor with respect to the elongate body. A fitting is coupled to the proximal end of the delivery pin and is configured to receive the tip of a medical agent delivery device.

For purposes of summarizing the invention and the advantages achieved over the prior art, certain objects and advantages of the invention have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the present invention herein disclosed. These and other embodiments of the present invention will become readily apparent to those skilled in the art from the following detailed description of the preferred embodiments having reference to the attached figures, the invention not being limited to any particular preferred embodiment(s) disclosed.

### BRIEF DESCRIPTION OF THE DRAWINGS

FIG. 1 is a posterior elevational posterior cross section through the proximal portion of the femur, illustrating two femoral neck fracture fixation devices positioned therein.

FIG. 2 is a side perspective view of a fixation device similar to that of FIG. 1.

FIG. 3 is a side elevational view of the fixation device of FIG. 2.

FIG. 4 is a cross-sectional view taken through line 4-4 of FIG. 3.

FIG. 4A is an enlarged view of portion 4A of FIG. 4.

FIG. 4B is a perspective view of a proximal anchor.

FIG. 4C is a side elevational view of the proximal anchor of FIG. 4B.

FIG. 4D is another side elevational view of the proximal anchor of FIG. 4B.

FIG. 4E is a frontal view of the proximal anchor of FIG. 4B.

FIG. 4F is a back view of the proximal anchor of FIG. 4B.

FIG. 4G is a cross-sectional side view of the proximal anchor of FIG. 4B.

FIG. 5 is a cross-sectional view taken through line 5-5 of FIG. 3.

FIGS. 6A-C illustrate a procedure for using of the fixation device of FIG. 1 to secure a femoral neck fracture.

FIG. 7 is a side view elevational view of an apparatus for delivering a bone fixation device and bone treatment agent.

FIG. 8 is a cross-sectional view of the apparatus of FIG. 7.

FIG. 8A is an enlarged view of a proximal end of the driver of FIG. 8, a deliver pin extends from the proximal end of the driver.

FIG. 8B is an enlarged view of a proximal anchor and a delivery pin coupled to a distal anchor of the apparatus of FIG. 8. The proximal anchor surrounds the junction of the delivery pin and the distal anchor.

FIG. 9 is a posterior elevational posterior cross section through the proximal portion of the femur, illustrating a bore extending through the femur and a guidewire positioned within the bore.

FIGS. 10-14 illustrate a procedure for using of a fixation device and delivery device to secure a femoral neck fracture and deliver a bone treatment agent.

FIG. 15 is a perspective view of another embodiment of an apparatus for delivering a bone fixation device and a bone treatment agent.

FIG. 16 is a cross-sectional view of the apparatus of FIG. 15.

FIG. 17 illustrates the fixation device and the apparatus of FIG. 15 securing a femoral neck fracture.

### DETAILED DESCRIPTION OF THE PREFERRED EMBODIMENTS

Although the delivery systems and/or fixation devices of the present invention will be disclosed primarily in the context of delivering bone treatment material (e.g., a medical or orthobiologic agent) to fractures of the proximal femur, the methods and structures disclosed herein are intended for application in any of a wide variety of bones and fractures, as will be apparent to those of skill in the art in view of the disclosure herein. Additionally, the fixation devices and delivery assemblies of the disclosed embodiments can deliver bone treatment agents to one or more locations in a bone. The delivery system of the present invention is applicable in a wide variety of fractures and osteotomies in the hand, such as interphalangeal and metacarpophalangeal arthrodesis, transverse phalangeal and metacarpal fracture fixation, spiral phalangeal and metacarpal fracture fixation, oblique phalangeal and metacarpal fracture fixation, intercondylar phalangeal and metacarpal fracture fixation, phalangeal and metacarpal osteotomy fixation as well as others known in the art. A wide variety of phalangeal and metatarsal osteotomies and fractures of the foot may also be stabilized and treated using the bone fixation device of the present invention. These include, among others, distal metaphyseal osteotomies such as those described by Austin and Reverdin-Laird, base wedge osteotomies, oblique diaphyseal, digital arthrodesis as well as a wide variety of others that will be known to those of skill in the art. The bone fixation device may be used with or without plate(s) or washer(s), all of which can be either permanent, absorbable, or combinations.

Fractures of the fibular and tibial malleoli, pilon fractures and other fractures of the bones of the leg may be fixated and stabilized with the present invention with or without the use of plates, both absorbable or non-absorbing types, and with alternate embodiments of the current invention. Fractures and osteotomies of the mid and hind foot, tarsal arthrodesis and osteotomy, or others as are known to those with skill in the art. One example is the fixation of the medial malleolar avulsion fragment. The delivery system can deliver bone treatment agents that may aid in the healing and/or the fixation and stabilization of the fractured bone.

The fixation device of the delivery system of the present invention may also be used to attach tissue or structure to the bone, such as in ligament reattachment and other soft tissue attachment procedures. Plates and washers, with or without tissue spikes for soft tissue attachment, and other implants may also be attached to bone, using either resorbable or nonresorbable fixation devices depending upon the implant and procedure. The fixation device may also be used to attach sutures to the bone, such as in any of a variety of tissue suspension procedures. The delivery system can deliver treatment agents to the tissue that may promote healing or provide desired physical characteristics.

For example, peripheral applications for the fixation devices include utilization of the device for fastening soft tissue such as capsule, tendon or ligament to bone. It may also be used to attach a synthetic material such as marlex mesh, to bone or allograft material such as tensor fascia lata, to bone. In the process of doing so, retention of the material to bone may be accomplished with the collar as shown, or the pin and or collar may be modified to accept a suture or other material for facilitation of this attachment.

Specific examples include attachment of the posterior tibial tendon to the navicular bone in the Kidner operation. This application may be accomplished using an appropriately sized implant of the present invention along with a washer with distally extending soft tissue spikes. Navicular-cuneiform arthrodesis may be performed utilizing the device and concurrent attachment of the tendon may be accomplished. Attachment of the tendon may be accomplished in the absence of arthrodesis by altering the placement of the implant in the adjacent bone.

Ligament or capsule reattachment after rupture, avulsion or detachment, such as in the ankle, shoulder or knee can also be accomplished using the devices disclosed herein.

The fixation devices may be used in combination with semi tubular, one-third tubular and dynamic compression plates, both of metallic and absorbable composition, if the collar is modified to match the opening on the plate.

The cannulated design disclosed below can be fashioned to accept an antibiotic impregnated rod for the slow adsorption of medication locally. This may be beneficial for prophylaxis, especially in open wounds, or when osteomyelitis is present and stabilization of fracture fragments is indicated.

A kit may be assembled for field use by military or sport medical or paramedical personnel. This kit contains an implanting tool, and a variety of implant device size and types. The kit may include additional components such as sterilization or disinfectant materials, a skin stapler, bandages, gloves, and basic tools for emergent wound and fracture treatment. Antibiotic rods may be included for wound prophylaxis during transport.

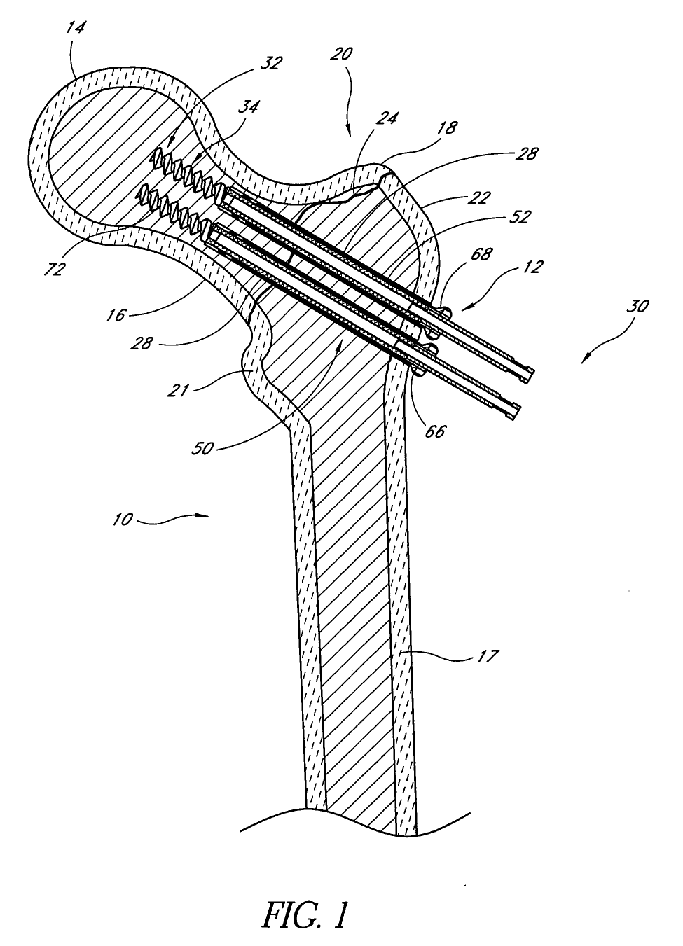

Referring to FIG. 1, there is illustrated a posterior side elevational view of the proximal portion of a femur 10, having a fixation device 12 positioned therein. The proximal end of the femur 10 comprises a head 14 connected by way of a neck 16 to the long body or shaft 17 of the femur 10. As illustrated in FIG. 1, the neck 16 is smaller in diameter than the head 14. The neck 16 and head 14 also lie on an axis which, on average in humans, crosses the longitudinal axis of the body 17 of the femur 10 at an angle of about 126°. The risk of fracture at the neck 16 is thus elevated, among other things, by the angular departure of the neck 16 from the longitudinal axis of the body 17 of femur 10 and also the reduced diameter of the neck 16 with respect to the head 14.

The greater trochanter 18 extends outwardly above the junction of the neck 16 and the body 17 of the femur 10. On the medial side of the greater trochanter 18 is the trochanteric fossa 20. This depression accommodates the insertion of the obturator extemus muscle. The lesser trochanter 21 is located posteromedially at the junction of the neck 16 and the body 17 of the femur 10. Both the greater trochanter 18 and the lesser trochanter 21 serve for the attachment of muscles. On the posterior surface of the femur 10 at about the same axial level as the lesser trochanter 21 is the gluteal tuberosity 22, for the insertion of the gluteus maximus muscle. Additional details of the femur are well understood in the art and not discussed in further detail herein.

FIG. 1 illustrates a fracture 24 which crosses the femur approximately in the area of the greater trochanter 18. Fractures of the proximal portion of the femur 10 are generally classified as capital or subcapital femoral neck fractures, intertrochanteric fractures and subtrochanteric fractures. All of these fractures will be deemed femoral neck fractures for the purpose of describing the present invention.

Referring to FIGS. 1-4, the fixation device 12 comprises a body 28 extending between a proximal end 30 and a distal end 32. The length, diameter and construction materials of the body 28 can be varied, depending upon the intended clinical application. In embodiments optimized for various fractures in an adult human population, the body 28 will generally be within the range of from about 10 mm to about 150 mm in length after sizing, and within the range of from about 2 mm to about 8 mm in maximum diameter. The major diameter of the helical anchor, discussed below, may be within the range of from about 2.7 mm to about 12 mm. In general, the appropriate dimensions of the body 28 will vary, depending upon the specific fracture. In rough terms, for a malleolar fracture, shaft diameters in the range of from about 3 mm to about 4.5 mm may be used, and lengths within the range of from about 25 mm to about 70 mm. For condylar fractures, shaft diameters within the range of from about 3.5 mm to about 6.5 mm may be used with lengths within the range of from about 25 mm to about 70 mm. For colles fractures (distal radius and ulna), diameters within the range of from about 2.0 mm to about 4.5 mm may be used with any of a variety of lengths within the range of from about 6 mm to about 70 mm.

In one embodiment, the body 28 comprises titanium. However, as will be described in more detail below, other metals or bioabsorbable or nonabsorbable polymeric materials may be utilized, depending upon the dimensions and desired structural integrity of the finished fixation device 12.

The distal end 32 of the body 28 is provided with a cancellous bone anchor or distal cortical bone anchor 34. Additional details of the distal bone anchor are described below. In general, in a femoral neck application, distal bone anchor 34 is adapted to be rotationally inserted into the cancellous bone within the head 14 of the femur 10, to retain the fixation device 12 within the femoral head.

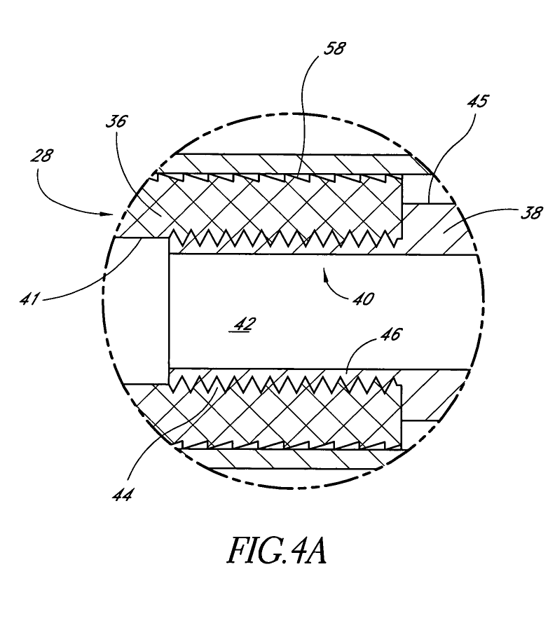

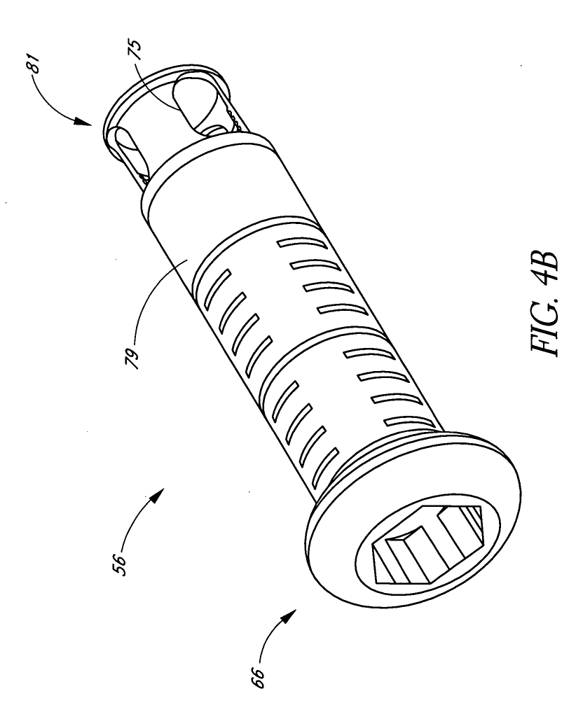

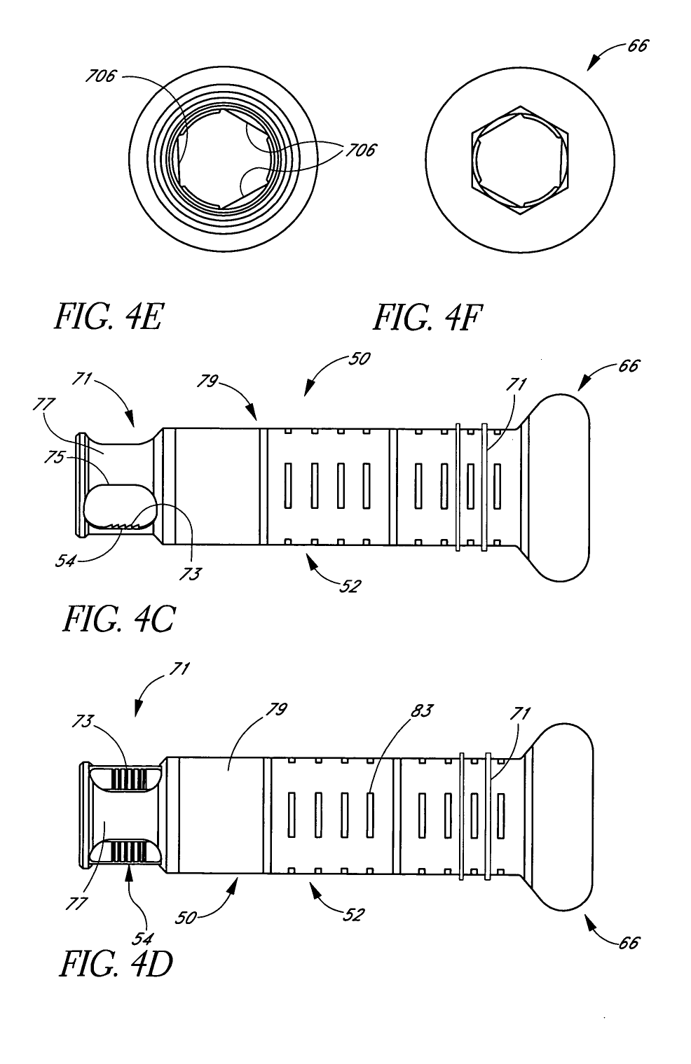

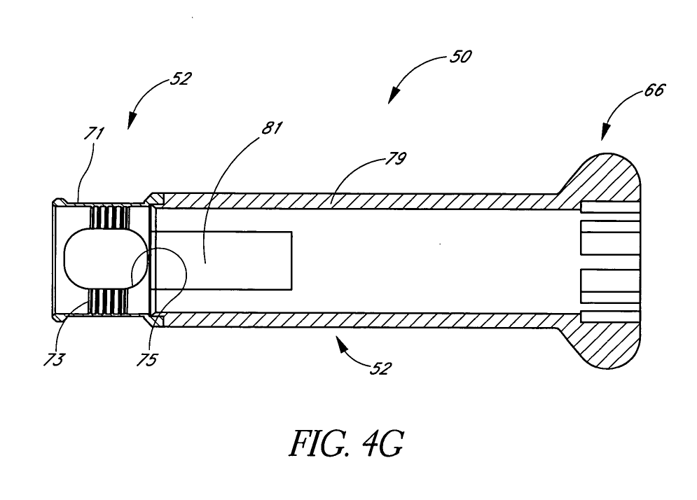

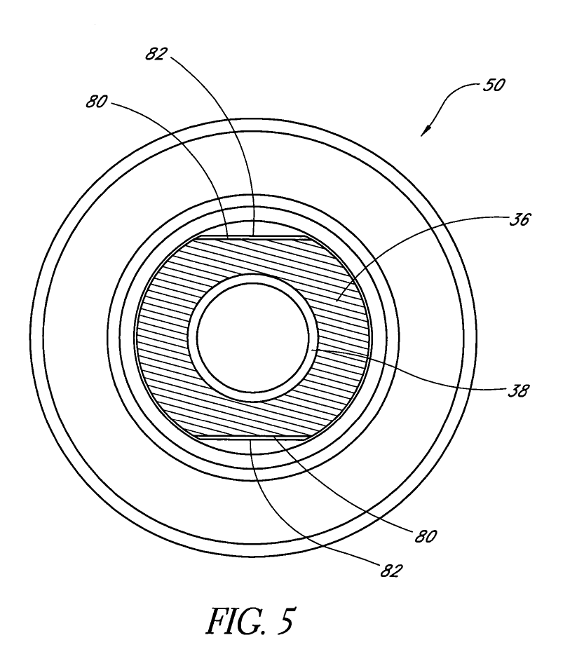

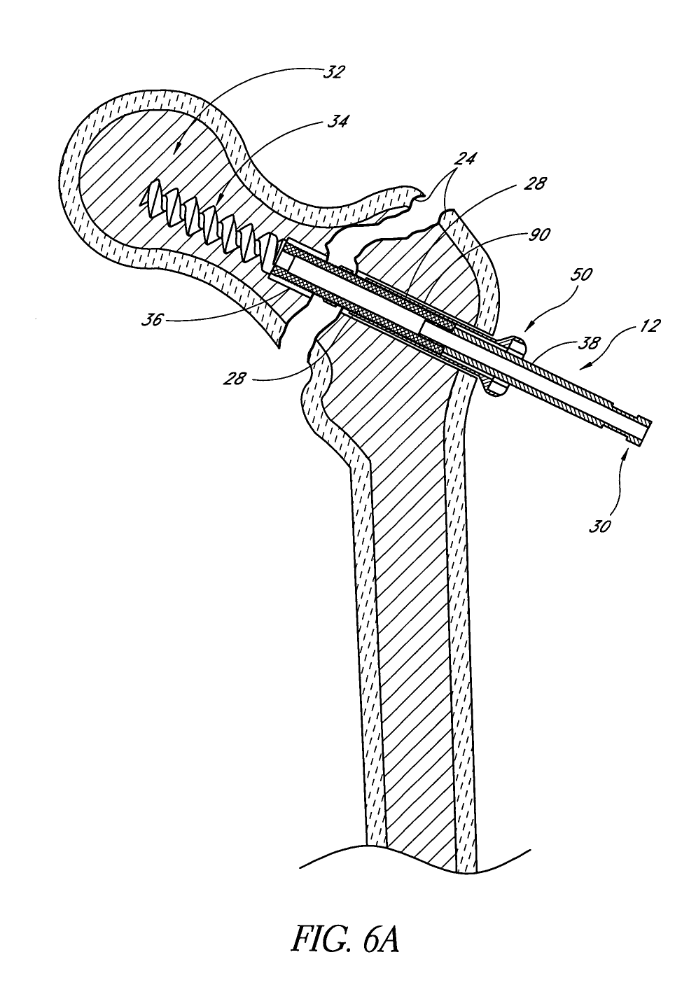

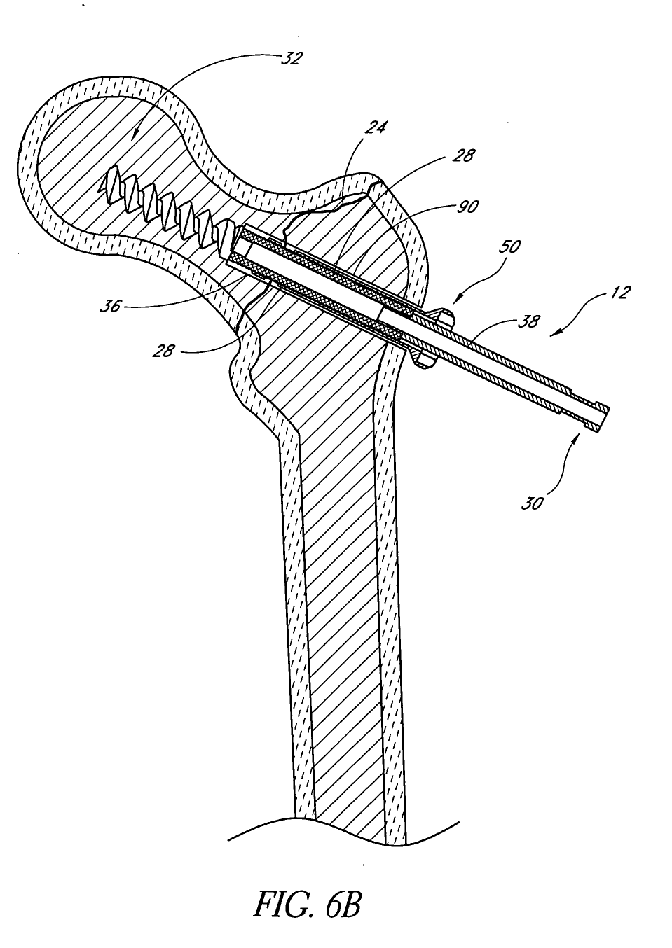

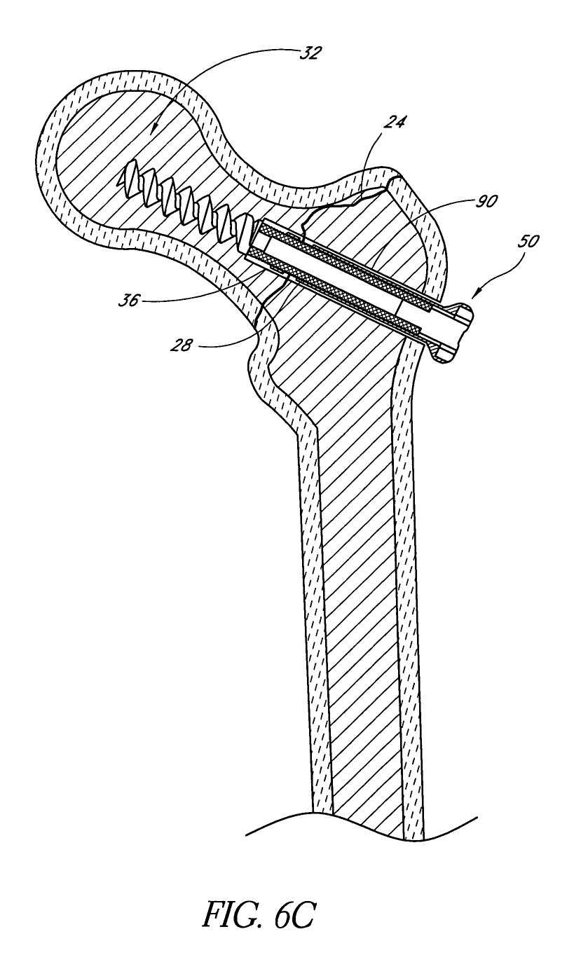

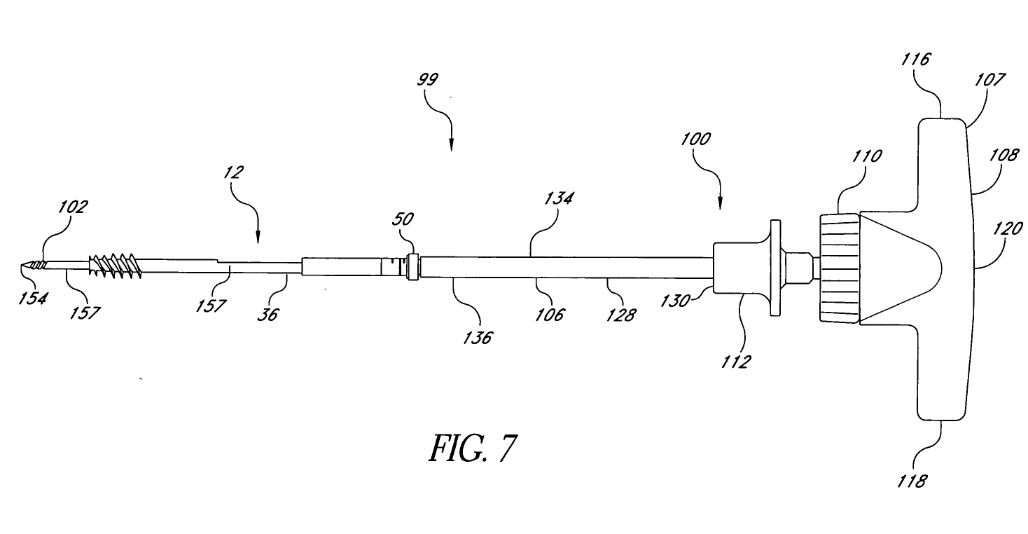

Referring to FIGS. 3, 4, and 4A, the body 28 comprises a first portion 36 and a second portion 38 that are coupled together at a junction 40. In the illustrated embodiment, the first portion 36 carries the distal anchor 34 while the second portion 38 forms the proximal end 30 of the body 28. The first and second portions 36, 38 are preferably detachably coupled to each other at the junction 40. In the illustrated embodiment, the first and second portions 36, 38 are detachably coupled to each other via interlocking threads. Specifically, as best seen in FIG. 4A, the body 28 includes an inner surface 41, which defines a central lumen 42 that preferably extends from the proximal end 30 to the distal end 32 throughout the body 28. In one embodiment, the central lumen 42 includes a first lumen 43 that extends through at least a portion of the first portion 36 and a second lumen 45 that extends through at least a portion of the second portion 38.

At the proximal end of the first portion 36, the inner surface 41 includes a first threaded portion 44. The first threaded portion 44 is configured to mate with a second threaded portion 46, which is located on the outer surface 45 of the second portion 38. The interlocking annular threads of the first and second threaded portions 44,. 46 allow the first and second portions 36, 38 to be detachably coupled to each other. In one modified embodiment, the orientation of the first and second threaded portions 44, 46 can be reversed. That is, the first threaded portion 44 can be located on the outer surface of the first portion 36 and the second threaded portion 46 can be located on the inner surface 41 at the distal end of the second portion 38. Any of a variety of other releasable complementary engagement structures may also be used, to allow removal of second portion 38 following implantation, as is discussed below.

In a modified arrangement, the second portion 38 can comprise any of a variety of tensioning elements for permitting proximal tension to be placed on the distal anchor 34 while the proximal anchor is advanced distally to compress the fracture. For example, any of a variety of tubes or wires can be removably attached to the first portion 36 and extend proximally to the proximal handpiece. In one such arrangement, the first portion 36 can include a releasable connector in the form of a latching element, such as an eye or hook. The second portion 38 can include a complementary releasable connector (e.g., a complementary hook) for engaging the first portion 36. In this manner, the second portion 38 can be detachably coupled to the first portion 36 such proximal traction can be applied to the first portion 36 through the second portion as will be explained below. Alternatively, the second portion 48 may be provided with an eye or hook, or transverse bar, around which or through which a suture or wire may be advanced, both ends of which are retained at the proximal end of the device. Following proximal tension on the tensioning element during the compression step, one end of the suture or wire is released, and the other end may be pulled free of the device. Alternate releasable proximal tensioning structures may be devised by those of skill in the art in view of the disclosure herein.

The proximal end 30 of the fixation device is provided with a proximal anchor 50. Proximal anchor 50 is axially distally moveable along the body 28, to permit compression of the fracture 24 as will be apparent from FIG. 1 and the description below. As will be explained below, complimentary locking structures such as threads or ratchet like structures between the proximal anchor 50 and the body 28 resist proximal movement of the anchor 50 with respect to the body 28 under normal use conditions. The proximal anchor 50 preferably can be axially advanced along the body 28 without rotation as will be apparent from the disclosure herein.

In the illustrated embodiment, proximal anchor 50 comprises a housing 52 such as a tubular body, for coaxial movement along the body 28. As best seen in FIGS. 1 and 4, in a final position, the housing 52 extends distally past the junction 40 between the first portion 36 and the second portion 38. The housing 52 is provided with one or more surface structures 54 such as a radially inwardly projecting flange 73 (see FIGS. 4C and 4G), for cooperating with complementary surface structures 58 on the first portion 36 of the body 28. In the illustrated embodiment, the complimentary surface structures 58 comprise a series of annular ridges or grooves 60. The surface structures 54 and complementary surface structures 58 permit distal axial travel of the proximal anchor 50 with respect to the body 28, but resist proximal travel of the proximal anchor 50 with respect to the body 28.

For example, as best seen in FIG. 4G, the proximal end of the flange 73 is biased towards the longitudinal axis of the body 28. As such, when the proximal anchor 50 is urged proximally with respect to the body 28, the flange 73 engages the grooves or ridges 60 of the complementary surface structures 58. This prevents proximal movement of the proximal anchor 50 with respect to the body 28. In contrast, as best seen in FIGS. 4 and 4C, when the proximal anchor 50 is moved distally with respect to the body 28, the flange 73 can bend outwardly away from the body 28 and the ridges 60 so as to allow the proximal anchor 50 to move distally. Of course, those of skill in the art will recognize that there are a variety of other complementary surface structures, which permit one way ratchet like movement. For example, a plurality of annular rings or helical threads, ramped ratchet structures and the like for cooperating with an opposing ramped structure or pawl can also be used. In one embodiment, opposing screw threads are dimensioned to function as a ratchet.

Retention structures 58 are spaced axially apart along the body 28, between a proximal limit 62 and a distal limit 64. The axial distance between proximal limit 62 and distal limit 64 is related to the desired axial working range of the proximal anchor 50, and thus the range of functional sizes of the fixation device 12. Thus, the present invention provides a bone fixation device which can provide compression across a fracture throughout a range of motion following the placement of the distal anchor. The distal anchor may be positioned within the cancellous and/or distal cortical bone, and the proximal anchor may be distally advanced throughout a range to provide compression across the fracture without needing to relocate the distal anchor and without needing to initially locate the distal anchor in a precise position with respect to the proximal side of the bone. Providing a working range throughout which tensioning of the proximal anchor is independent from setting the distal anchor allows a single device to be useful for a wide variety of fractures, as well as eliminates the need for accurate device measurement and accurate placement of the distal anchor. In many applications, the working range is at least about 10% of the overall length of the device, and may be as much as 20% or 30% or more of the overall device length. In the context of a femoral application, working ranges of up to about 10 mm or more may be provided, since estimates within that range can normally be readily accomplished within the clinical setting. In other applications, such as a metatarsal fracture, a working range in the area of from about 1 mm to about 2 mm may be all that is necessary. The embodiments disclosed herein can be scaled to have a greater or a lesser working range, as will be apparent to those of skill in the art in view of the disclosure herein.

The proximal anchor 50 includes a flange 66 that seats against the outer surface of the femur or tissue adjacent the femur. The flange 66 is preferably an annular flange, to optimize the footprint or contact surface area between the flange 66 and the femur. Circular or polygonal shaped flanges for use in femoral head fixation will generally have a diameter of at least about 4 mm greater than the adjacent body 28 and often within the range of from about 4 mm to about 20 mm or more greater than the adjacent body 28.

In the illustrated embodiment, the bone contacting surface 68 of the flange 44 is tapered and generally faces the shaft 17 of the femur 10. In other embodiments, the bone contacting surface 68 can resides in or approximately on a plane, which is perpendicular with respect to the longitudinal axis of the body 28. In other embodiments, other angular relationships between the bone contacting surface 68 of the flange 66 and the longitudinal axis of the body 28 and housing 52 may be utilized, depending upon the anticipated entrance angle of the body 28 and associated entrance point surface of the femur 10. In general, the longitudinal axis extending through the head 14 and neck 16 of the human femur is inclined at an angle of approximately 126° from the longitudinal axis of the long body 17 of the femur 10. Angles between the longitudinal axis of body 28 and tissue contacting surface 68 within the range of from about 90° to about 140° will generally be utilized.

In a modified embodiment, the housing 52 of the proximal anchor 50 can include one or more one or more barbs that extend radially outwardly from the tubular housing 52. Such barbs provide for self tightening after the device has been implanted in the patient as described in a co-pending U.S. Pat. Nos. 6,908,465, 6,890,333 entitled “DISTAL BONE FOR BONE FIXATION WITH SECONDARY COMPRESSION” and U.S. Pat. No. 6,887,243, which are hereby expressly incorporated by reference herein. The barbs may be radially symmetrically distributed about the longitudinal axis of the housing 52. Each barb is provided with a transverse engagement surface, for anchoring the proximal anchor 50 in the bone. The transverse engagement surface may lie on a plane which is transverse to the longitudinal axis of the housing 50 or may be inclined with respect to the longitudinal axis of the tubular 50. In either arrangement, the transverse engagement surface 43 generally faces the bone contacting surface 68 of the flange 44. As such, the transverse engagement surface inhibits proximal movement of the proximal anchor with respect to the bone. In the illustrated embodiment, for example, the proximal anchor 50 also has one or more ribs 71 (FIGS. 4C and 4D) that can engage the bone to reduce movement between the anchor 50 and the bone. The ribs may be a plurality of annular protrusions axially spaced along the exterior of the anchor 50. However, the ribs can be any protrusions, protuberances, texturing, or other surface treatment suitable to achieve the desired interaction between the bone the anchor 50.

The clinician can be provided an array of proximal anchors 50 of varying angular relationships between the bone contacting surface 68 and the longitudinal axis of the body 28 and housing 52 (e.g., 90°, 100°, 110°, 120°, and 130°). A single body 28 can be associated with the array such as in a single sterile package. The clinician upon identifying the entrance angle of the body 28 and the associated entrance point surface orientation of the femur 10 can choose the anchor 50 from the array with the best fit angular relationship, for use with the body 28.

With particular reference to FIGS. 3, the proximal end 30 of the body 28 may be provided with a rotational coupling 70, for allowing the second portion 38 of the body 28 to be rotationally coupled to a rotation device. The proximal end 30 of the body 28 may be desirably rotated to accomplish one or more discrete functions. In one application of the invention, the proximal end 30 is rotated to remove the second portion 38 of the body 28 following tensioning of the device across a fracture or to anchor an attachment to the bone. Rotation of the rotational coupling 70 may also be utilized to rotationally drive the distal anchor into the bone. Optionally, the rotational coupling 70 may be a connector configured to receive treatment material (e.g., orthobiological agents) as described below in detail. Any of a variety of rotation devices may be utilized, such as electric drills or hand tools, which allow the clinician to manually rotate the proximal end 30 of the body. Thus, the rotational coupling 70 may have any of a variety of cross sectional configurations, such as one or more flats or splines. Additionally, the coupling 70 may be a luer connector or other configuration designed to permit rotation of the second portion 38 and to receive treatment agent.

In one embodiment, the rotational coupling 70 comprises a proximal projection of the body 28 having an axial recess with a polygonal cross section, such as a hexagonal cross section. The rotational coupling 70 is illustrated as a female component, machined or milled or attached to the proximal end 30 of the body 28. However, the rotational coupling may also be in the form of a male element, such as a hexagonal or other noncircular cross sectioned projection.

As illustrated, the body 28 is cannulated to accommodate installation over a placement wire as is understood in the art. The cross section of the illustrated central cannulation is circular but in other embodiments may be non circular, e.g., hexagonal, to accommodate a corresponding male tool for installation or removal of the second portion 38 of the body 28 as will be explained below. In other embodiments, the body 28 may partially or wholly solid.

In all of the embodiments illustrated herein, the distal anchor 34 comprises a helical locking structure 72 for engaging cancellous and/or distal cortical bone. In the illustrated embodiment, the locking structure 72 comprises a flange that is wrapped around the axial lumen. The flange extends through at least one and generally from about two to about 50 or more full revolutions depending upon the axial length of the distal anchor and intended application. For most femoral neck fixation devices, the flange will generally complete from about 2 to about 20 revolutions. The helical flange 72 is preferably provided with a pitch and an axial spacing to optimize the retention force within cancellous bone, to optimize compression of the fracture.

The helical flange 72 of the illustrated embodiment has a generally triangular cross-sectional shape (see FIG. 4). However, it should be appreciated that the helical flange 72 can have any of a variety of cross sectional shapes, such as rectangular, oval or other as deemed desirable for a particular application through routine experimentation in view of the disclosure herein. The outer edge of the helical flange 72 defines an outer boundary. The ratio of the diameter of the outer boundary to the diameter of the central lumen can be optimized with respect to the desired retention force within the cancellous bone and giving due consideration to the structural integrity and strength of the distal anchor 34. Another aspect of the distal anchor 34 that can be optimized is the shape of the outer boundary and the central core, which in the illustrated embodiment are generally cylindrical.

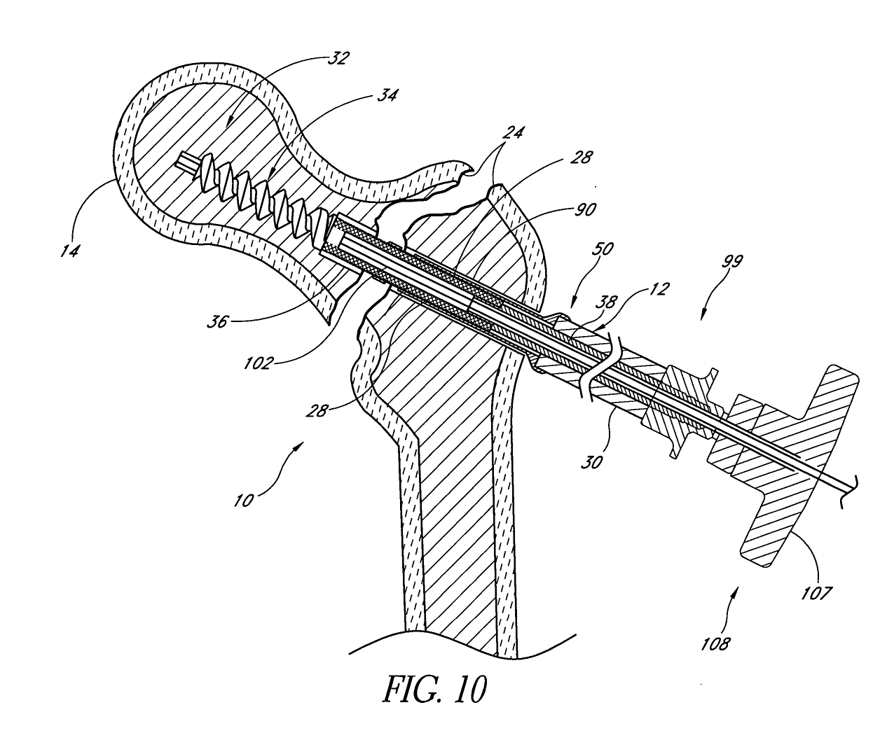

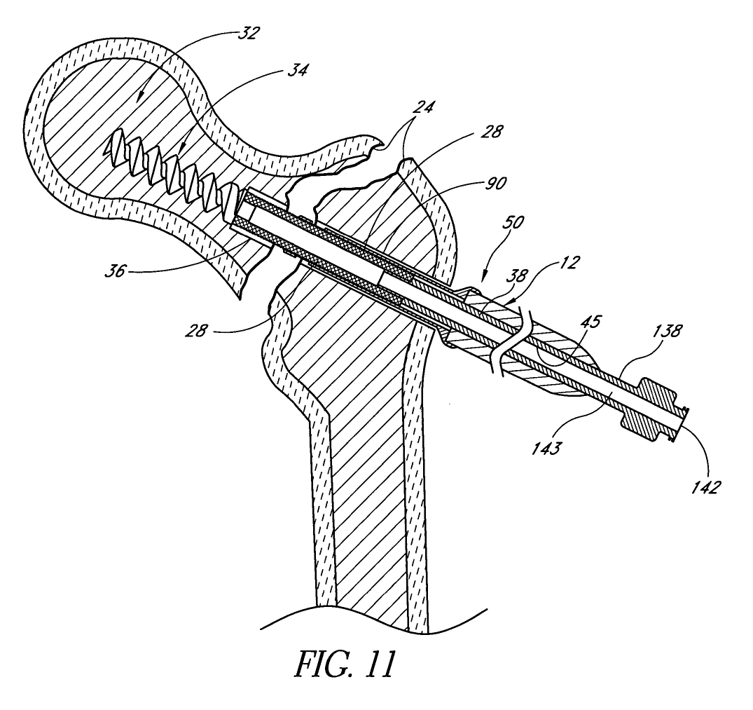

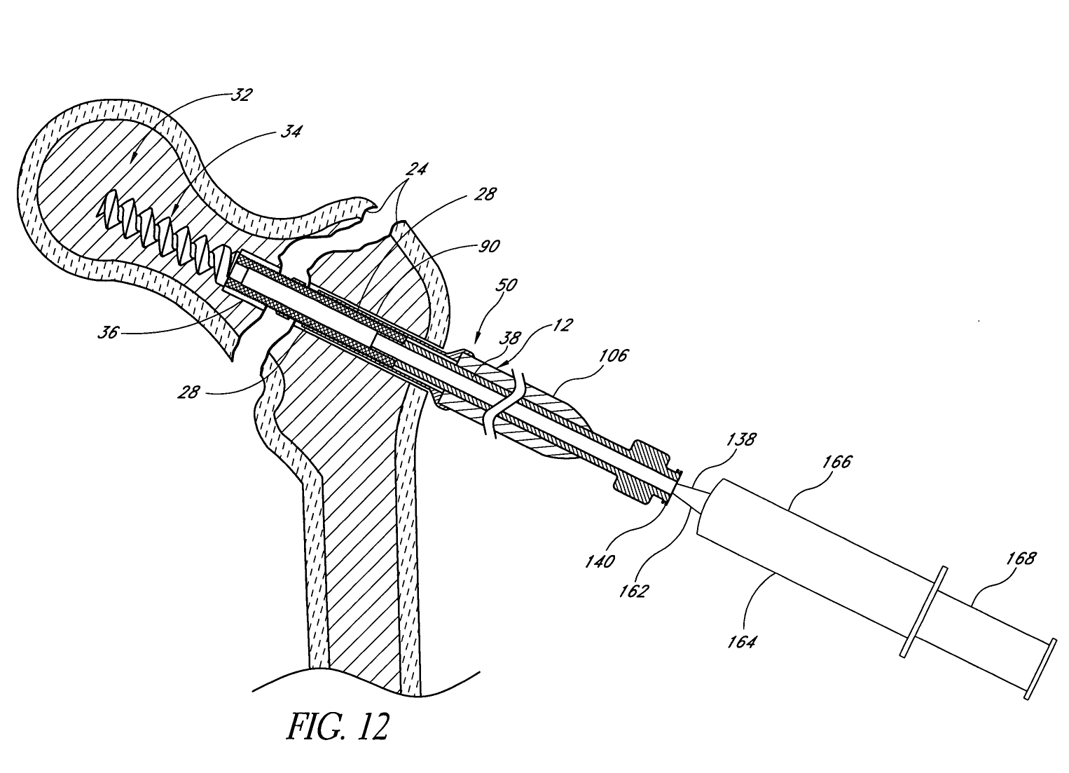

Optionally, the helical flange 72 may comprise threads spiraled about a tubular body as shown in FIG. 8. Thus, there may or may not be gaps between one or more of the threads of the helical flange 72. Preferably, the interior portion of the helical flange 72 defines a delivery path for treatment material. In the illustrated embodiment, the helical flange 72 may define a portion of the first lumen 43.

It is contemplated that the distal anchor 34 may comprises any suitable structure for engaging cancellous and/or distal cortical bone. For example, the one or more helical locking structures, such as the helical locking structure described above. In one embodiment, the distal anchor 34 comprises a double helix or other structure described in U.S. Pat. No. 6,887,243. The entire contents this patent is hereby expressly incorporated by reference. Preferably, treatment material can be delivered and deployed from the distal anchor 34.

The distal end 32 and/or the outer edges of the helical flange 72 may be atraumatic (e.g., blunt or soft). This inhibits the tendency of the fixation device 12 to migrate anatomically proximally towards the hip joint bearing surface after implantation (i.e., femoral head cut-out). Distal migration is also inhibited by the dimensions and the presence of the proximal anchor 50, which has a larger footprint than conventional screws. To further reduce migration of the fixation device 12, an agent or treatment material (e.g., bone cement) can be delivered from the fixation device 12 and into the bone proximate to at least a portion of the bone fixation device. The bone cement can set and inhibit, preferably prevent, migration of the fixation device 12.

A variety of other arrangements for the distal anchor 32 can also be used. For example, the various distal anchors described in U.S. Pat. No. 6,511,481 issued on Jan. 28, 2003, and co-pending U.S. patent application entitled “DISTAL BONE FOR BONE FIXATION WITH SECONDARY COMPRESSION”, filed Nov. 13, 2001 can be incorporated into the fixation device 12 described herein. The entire contents these applications are hereby expressly incorporated by reference. In particular, the distal anchor may comprise a single helical thread surrounding a central core, much as in a conventional screw, which has been cannulated to facilitate placement over a wire. Alternatively, a double helical thread may be utilized, with the distal end of the first thread rotationally offset from the distal end of the second thread. The use of a double helical thread can enable a greater axial travel for a given degree of rotation and greater retention force than a corresponding single helical thread. Specific distal anchor designs can be optimized for the intended use, taking into account desired performance characteristics, the integrity of the distal bone, and whether the distal anchor is intended to engage exclusively cancellous bone or will also engage cortical bone.

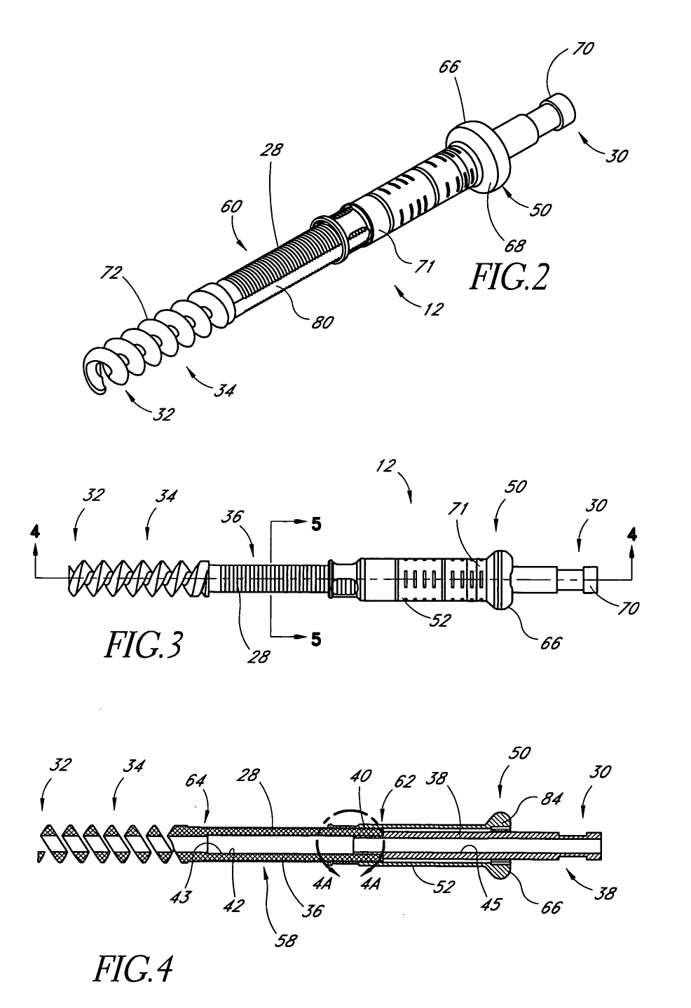

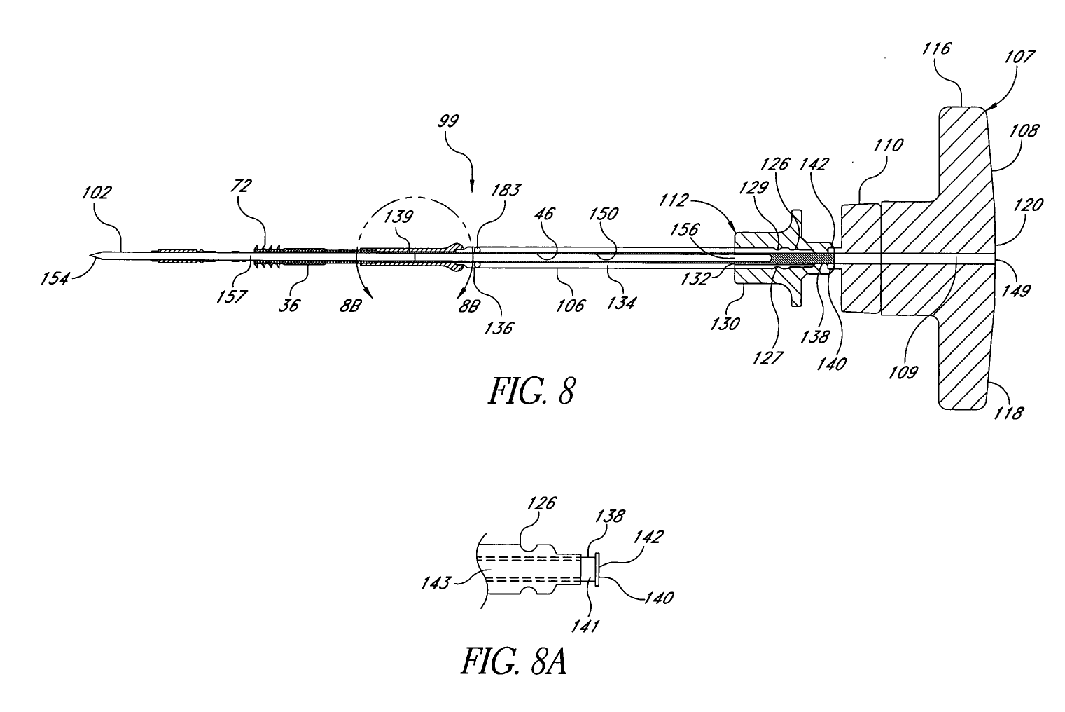

With particular reference to FIGS. 2 and 5, the fixation device may include an antirotation lock between the first portion 36 of the body 28 and the proximal anchor or collar 50. In the illustrated embodiment, the first portion 36 includes a pair of flat sides 80, which interact with corresponding flat structures 81 (FIG. 4G) in the proximal collar 50. One or three or more axially extending flats may also be used. As such, rotation of the proximal collar 50 is transmitted to the first portion 36 and distal anchor 34 of the body 28. Of course, those of skill in the art will recognize various other types of splines or other interfit structures can be used to prevent relative rotation of the proximal anchor and the first portion 36 of the body 28.

To rotate the proximal collar, the flange 66 is preferably provided with a gripping structure to permit an insertion tool or system to rotate the flange 66. Any of a variety of gripping structures may be provided, such as one or more slots, flats, bores or the like. In one embodiment, the flange 66 is provided with a polygonal, and, in particular, a pentagonal or hexagonal recess 84. See FIG. 4.

FIGS. 4G illustrate in more detail the proximal anchor 50 of FIGS. 2-4. This embodiment includes the tubular housing 51, which includes a tubular housing 71 includes an inner surface with one or more teeth or flanges 73 (FIG. 4E), which are configured to engage the grooves or ridges 60 on the body 28. One or more slots or openings 75 are formed in the tubular housing to form one or more bridges 77, which carry the grooves or ridges 73. The anchor 50 can be pushed towards the distal end of the body and the teeth can slide along and be lifted over the retention structures 60 of the body as the bridge is flexed away from the body. The number and shape of the openings and bridges may be varied depending of the desired flexing of the bridges when the proximal anchor is moved distally over the body and the desired retention force of the distal anchor when appropriately tensioned. In one embodiment, the teeth on the proximal anchor and the grooves on the body 28 may be configured such that the proximal anchor can be rotated or threaded onto the pin in the proximal direct and/or so that the proximal anchor can be removed by rotation.

In the illustrated embodiment, the tubular housing 71 is attached to, coupled to, or integrally formed (partially or wholly) with a secondary tubular housing 79, which includes one or more anti-rotational features 81 (e.g., flat sides) for engaging corresponding anti-rotational features formed on the pin. The flange or collar 66 is attached, coupled or integrally formed with the proximal end of the secondary tubular housing. The teeth or flanges 73 on the bridges 77 may also be configured such that the proximal anchor may be distally advanced and/or removed with rotation. The illustrated embodiment of the housing 79 also advantageously includes visual indicia 83 (e.g., marks, grooves, ridges etc.) on the tubular housing 79 for indicating the depth of the proximal anchor 50 within the bone. Thus, the housing 51 can comprise one or more housings that are coupled to each other and/or integrally formed.

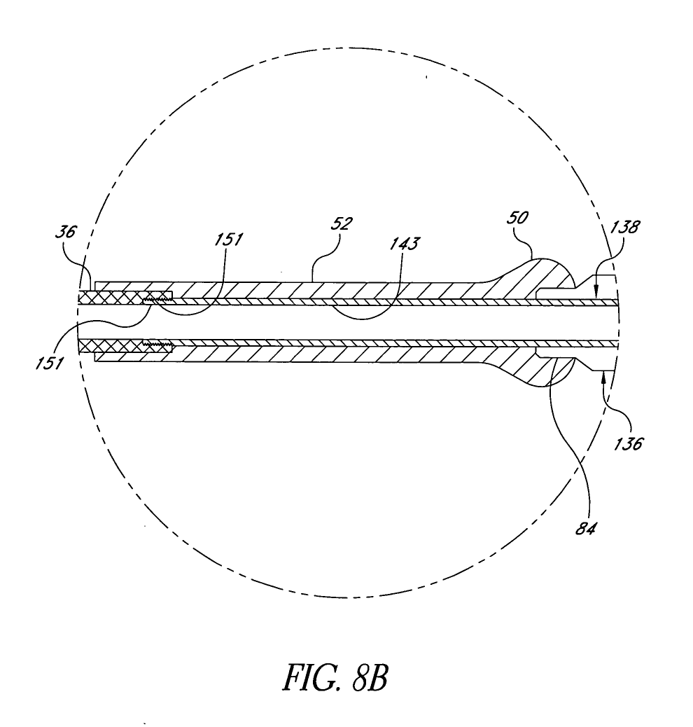

In use, the clinician first identifies a patient having a fracture to be treated, such as a femoral neck fracture, which is fixable by an internal fixation device. The clinician accesses the proximal femur, reduces the fracture if necessary and selects a bone drill and drills a hole 90 (see FIG. 6A) in accordance with conventional techniques. Frequently, the hole 90 has a diameter within the range from about 3 mm to about 8 mm. This diameter may be slightly larger than the diameter of the distal anchor 34. The hole 90 preferably extends up to or slightly beyond the fracture 24.

In one embodiment of use, a fixation device 12 having an axial length and outside diameter suitable for the hole 90 is selected. The distal end 32 of the fixation device 12 is advanced distally into the hole 90 until the distal anchor 34 reaches the distal end of the hole 90. The proximal anchor 50 may be carried by the fixation device 12 prior to advancing the body 28 into the hole 90, or may be attached following placement of the body 28 within the hole 90. Once the body 28 and proximal anchor 50 are in place, the clinician may use any of a variety of driving devices, such as electric drills or hand tools to rotate the proximal anchor 50 and thus cancellous bone anchor 34 into the head of the femur. In modified embodiments, the fixation device is configured to be self-drilling or self tapping such that a hole does not have to be formed before insertion into the bone.

Once the anchor 34 is in the desired location, proximal traction is applied to the proximal end 30 of body 28, such as by conventional hemostats, pliers or a calibrated loading device, while distal force is applied to the proximal anchor 50. In this manner, the proximal anchor 50 is advanced distally until the anchor 50 fits snugly against the outer surface of the femur or tissue adjacent the femur and the fracture 24 is completely reduced as shown in FIG. 6B. Appropriate tensioning of the fixation device 12 is accomplished by tactile feedback or through the use of a calibration device for applying a predetermined load on the implantation device. One advantage of the structure of the present invention is the ability to adjust compression independently of the setting of the distal anchor 34.

Following appropriate tensioning of the proximal anchor 50, the second portion 38 of the body 28 is preferably detached from the first portion 36 and removed. See FIG. 6C. In the illustrated embodiment, this involves rotating the second portion 38 with respect to the first portion via the coupling 70. In connection with many of the fractures identified previously herein, a single fixation device 12 may be all that is clinically indicated. However, two or three or more fixation devices 12 may be utilized to reduce a single fracture, depending upon the location and physical requirements of the fractured portion of the bone. For example, in the case of proximal femoral fractures of the type illustrated herein, typically at least two and preferably three fixation devices 12 will be implanted to span the femoral neck. The use of three fixation devices 12 desirably provides sufficient compression across the fracture, as well as minimizes the risk of rotation of the head of the femur around the axis of a single fixation device 12. The proximal end of the fixation devices may be connected together such as through a three-holed plate or rod, or may be independent of each other.

An advantage certain embodiments of the fixation devices disclosed above is that the proximal anchor provides the device with a working range such that one device may accommodate varying distances between the distal anchor and the proximal anchor. In certain applications, this allows the technician to focus on the proper positioning of the distal anchor with the knowledge that the proximal anchor lies within the working range of the device. With the distal anchor positioned at the desired location, the proximal anchor may then be advanced along the body to compress the fracture and/or provide stability between bones. In a similar manner, the working range provides the technician with flexibility to adjust the depth of the proximal anchor. For example, in some circumstances, the bone may include voids, cysts osteoporotic bone that impairs the stability of the distal anchor in the bone. Accordingly, in some circumstances, the technician may advance the distal anchor and then desire to retract the distal anchor such that it is better positioned in the bone. In another circumstance, the technician may inadvertently advance the distal tip through the bone into a joint space. In such circumstances, the working range of the device allows the technician to reverse and retract the anchor and recompress connection. Such adjustments are facilitated by the working range of the proximal anchor on the body.

Following removal of the second portion 38 of each body 28, the access site may be closed and dressed in accordance with conventional wound closure techniques.

In a modified arrangement, the second portion 38 may form part of the driving device, which is used to rotate the proximal anchor 50 and thus cancellous bone anchor 34 into the head of the femur. The second portion 38 is used to apply proximal traction so as to compress the fracture. After appropriate tensioning, the second portion 38 can be de-coupled from the first portion 36 and removed with the driving device.

In the foregoing variation, the second portion 38 may be connected to a rotatable control such as a thumb wheel on the deployment device. A container may be opened at the clinical site exposing the proximal end of the implant, such that the distal end of the second portion 38 may be removably coupled thereto. Proximal retraction of the hand tool will pull the implant out of its packaging. The implant may then be positioned within the aperture in the bone, rotated to set the distal anchor, and the hand piece may be manipulated to place proximal traction on the second portion 38 while simultaneously distally advancing the proximal anchor. Following appropriate tensioning across the fracture, the second portion 38 may be disengaged from the implant, and removed from the patient. In the example of a threaded engagement, the second portion 38 may be disengaged from the implant by rotating a thumb wheel or other rotational control on the hand piece. In an alternate embodiment, such as where the second portion 38 comprises a pull wire, following appropriate tensioning across the fracture, a first end of the pull wire is released such that the pull wire may be removed from the implant by proximal retraction of the second end which may be attached to the hand piece.

Preferably, the clinician will have access to an array of fixation devices 12, having, for example, different diameters, axial lengths and, if applicable, angular relationships. These may be packaged one per package in sterile envelopes or peelable pouches, or in dispensing cartridges which may each hold a plurality of devices 12. Upon encountering a fracture for which the use of a fixation device is deemed appropriate, the clinician will assess the dimensions and load requirements, and select a fixation device from the array, which meets the desired specifications.

In some instances, a clinician may want to introduce two or more fixation devices 12 into the femoral head 14 to secure the fracture 24. This may be desirable if the clinician determines that, based upon the nature of the fracture 24, there is a possibility that the head 14 of the femur 10 could rotate about a single fixation device 12. Even minor rotation can inhibit the healing of the fracture. Significant rotation can result in failure of the fixation device or necrosis of the femoral head. Two or more fixation devices 12 may also be desirable where the direction of the fracture is generally parallel to the axis of implantation as is understood in the art.

The fixation device 12 of the present invention may also be used in combination with intramedullary nails or rods, as will be understood by those of skill in the art.

The fixation device 12 of the present invention may be used in any of a wide variety of anatomical settings beside the proximal femur, as has been discussed. For example, lateral and medial malleolar fractures can be readily fixed using the device of the present invention.

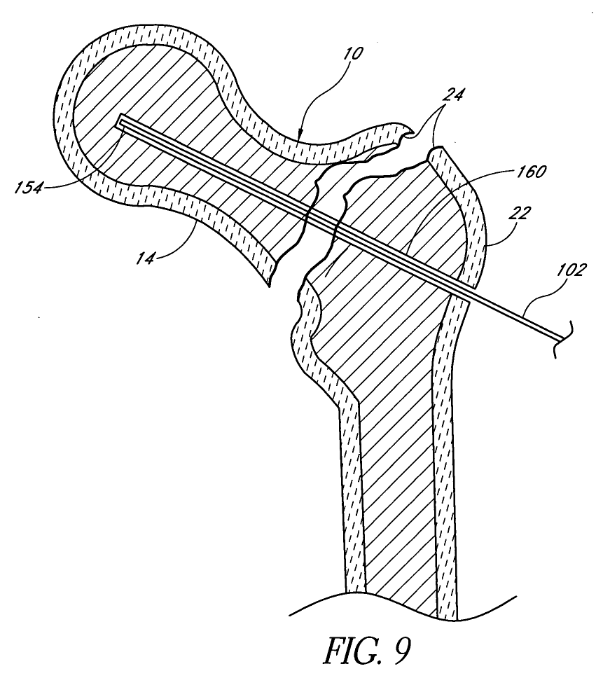

FIGS. 7 and 8 illustrate an exemplary system 99 for delivering a treatment agent within a bone. The treatment agent may be any of a variety of treatment agents that are used to promote healing and/or fixation during a bone fixation or fusion procedures. Such agents include, but are not limited, to orthobiologics and bone cements. Orthobiologics refer generally to treatment agents that are used to replace, repair, and/or regenerate damaged tissue and/or bones. Osteoinductives, also known as growth factors, are a type of orthobiologics that are configured for regenerating bone. For example, they may include proteins that activate the formation of new bone. Another type of orthobiologic are osteoconductive bone replacement materials, which assist bone regeneration by providing a scaffold that supports the ingrowth of capillaries and provides a host bed for new cells. Such osteoconductive materials may be resorbable or non-resorbable. In a similar manner, bone cement maybe use to supplement internal fixation. In particular, bone cement may be used to strengthen osteoporotic or otherwise weakened bone to enhance the holding characteristic of the distal anchor. The treatment agents described above may also be combined into various combinations. For example, bone cement may be impregnated with a growth factor to form a particularly affective treatment agent.

With initial reference to FIG. 7, the exemplary system 99 generally comprises an insertion device or tool 100, which may be coupled to a fixation device 12 configured as described above. As will be explained below, the insertion device 100 and fixation device 12 may be inserted over a guidewire 102. Generally, the insertion device 100 is configured to engage with the proximal anchor 50 of the fixation device 12, such that the insertion device 100 can be used to rotate the fixation device 12 into the desired position. After positioning, the deployment device 100 can be disengaged and separated from the fixation device 12. A medical agent may then be injected into bone through the central lumen of the system 99 as described below.

With reference to FIGS. 7 and 8, the insertion device 100 generally comprises an elongated drive rod 106 and a handle assembly 107. The handle assembly 107 generally comprises a handle 108 and a distal hub 112. The illustrated handle 108 is T-shaped with a pair of side portions 116, 118 and a central portion 120 configured to engage the clinician's palm. The handle 108 is configured so that the clinician can conveniently grip the handle 108 and rotate the insertion device 100 as described below. The handle 108 may include a through lumen 109, which may be used as described below for delivering a medical agent through the fixation device 12.

The illustrated handle assembly 107 includes knurled region 110 that is positioned distally of the central portion 120. The knurled region 110 may be coupled to or integrally formed with the distal hub 112. As shown in FIG. 8, in the illustrated embodiment, the lumen 109 extends through the handle 108, the knurled region 110, and the distal hub 112. The lumen 109 may form in part a socket 124, which as will be explained below, is configured to releasably receive a proximal end 126 of the driver 106 and to cause rotation of the driver 106.

With reference to FIGS. 8 and 8A, the driver 106 comprises an elongated body 134 having the proximal end 126 and a distal end 136. As mentioned above, the proximal end 126 of the driver 106 is configured to be gripped within the socket 124 of the handle assembly 107. In one embodiment, the proximal end 126 is hexagonal in shape and the socket 124 has a corresponding shape for receiving the proximal end 126 of the driver. However, it should be appreciated that the proximal end 126 and socket 124 can have any of a variety of different shapes for transmitting rotation from the handle assembly 107 to the driver 106. For example, the proximal end 126 and socket 124 can have one or more flat sides that are polygonal in shape. In still other embodiments, the proximal end 126 may comprise a recess configured to engage an anti-rotational protrusion formed on the socket 124.

The socket 124 is advantageously also configured to releasably engage the proximal end 126 of the driver 106 to prevent axial relative movement between the handle assembly 107 and the driver 106. Accordingly, in the illustrated embodiment, the socket 124 includes a protrusion 127 that is configured to engage a corresponding recess 129 in the proximal end 126 of the driver 106. The protrusion 127 and the recess 129 cooperate to prevent axial relative movement between the distal hub 112 and the driver 106. In one embodiment, there is snap or interference fit between the socket 124 and the proximal end 126. In this manner, the driver 106 can be easily and conveniently snapped into and out of the distal hub 112. Of course in other embodiments, pins, ties, screws, fasteners, and/or other configurations can be used to releasably couple the driver 106 to the handle 107. In still other embodiments, the driver 106 and handle 107 may be provided without the realeable coupling described above.

With reference to FIGS. 8 and 8B, the distal end 136 of the driver 106 may include an outer portion configured to engage the gripping structure of the proximal anchor 50. As explained above, in the illustrated embodiment, the gripping structure comprises a hexagonal recess 84. Accordingly, the distal end 136 of the illustrated embodiment has a hexagonal protrusion that is configured to be received by the hexagonal recess 84 of the proximal anchor 50. In other embodiments, the distal end 136 may have any of a variety of different shapes for differently shaped gripping structures 84 on the proximal anchor 50. For example, the distal end 136 can have one or more slots, flats, bores, recesses or the like that are configured to engage the gripping structure 84 of the proximal anchor 50.

From the above description, it should be apparent that rotation of the handle assembly 107 is transmitted through the driver rod 106 to the proximal anchor 50 of the fixation device 12. The elongated body 134 of the driver 106 may have a generally tubular shape as shown in the illustrated embodiment and is configured to withstand loads applied to the deployment device 100 by the clinician. As shown in FIG. 8, the elongated body 134 defines the lumen 150 having a generally uniform cross-sectional profile along its length. However, in other embodiments, the elongated body 134 and/or lumen 150 can have a cross-sectional profile that varies along its length. The size and configuration of the elongated body 134 can be determined based on the desired loads (e.g., torque and/or axial loads) applied to the fixation device 12. For example, the wall thickness of the elongated body 134 can be increased to accommodate a high torque that is applied to the fixation device 12.

The lumen 150 is generally configured to receive a proximal end of a delivery pin 138, which will be described below. Optionally, an O-ring 183 can be disposed along the lumen 150. The O-ring 183 can inhibit or prevent substances (e.g., blood, tissue, or the like) from passing between the delivery pin 138 and the driver 106. Preferably, the O-ring 183 permits rotational movement between the delivery pin 138 and the driver 106.

With continued reference to FIGS. 8, 8A and 8B, the delivery pin 138 comprises a distal end 139 and a proximal end 142 and a lumen 143 extending therethrough. In the illustrated embodiment, the delivery pin 138 extends from the handle 107 to the first portion 36. The distal end 139 is configured to engage the first portion 36 of the body of the fixation device 12 in a manner similar to the second portion 38 described above. Accordingly, the distal end 139 includes a complementary releasable connector for engaging the first portion 36. In the illustrated embodiment, the complementary releasable connector 151 (FIG. 8B) comprises a threaded portion formed on the outer surface of the distal end 139 of the delivery pin 138. The lumen 143 of the delivery pin 138 is configured to extend over the guide wire 102. Thus, it is contemplated that the delivery pin 138 can have a different or similar distal structure as the second portion 38 for interacting with the first portion 36.

As shown in FIG. 8 and 8A, the body of the delivery pin 138 preferably extends through the lumen 150 of the driver rod 106 such that the proximal end 142 of the delivery pin 138 extends past the proximal end 126 of the driver rod 106. The proximal end 142 of the pin 138 preferably includes a fitting 141, which as will be explained below is configured to mate with a delivery device for delivering a medical agent. In the illustrated embodiment, the fitting comprises a flange 140 that is sized and configured such that the end of a syringe can be conveniently inserted and advanced into the fitting 141. When the handle assembly 107 is removed from the driver rod 106, the fitting 141 is preferably exposed. In this manner, a medical agent (e.g., an orthobiologic agent, cement, growth factor, and/or the like) may be injected into the lumen 143 from a syringe, bag, line, and/or any other suitable device for delivering an treatment agent. Preferably, the guidewire 102 is removed before the medical agent is injected into the fixation device 12. As will be explained in more detail below, in a modified embodiment the delivery pin 138 may extend through the handle assembly 107, such that the fitting extends proximally from the handle.

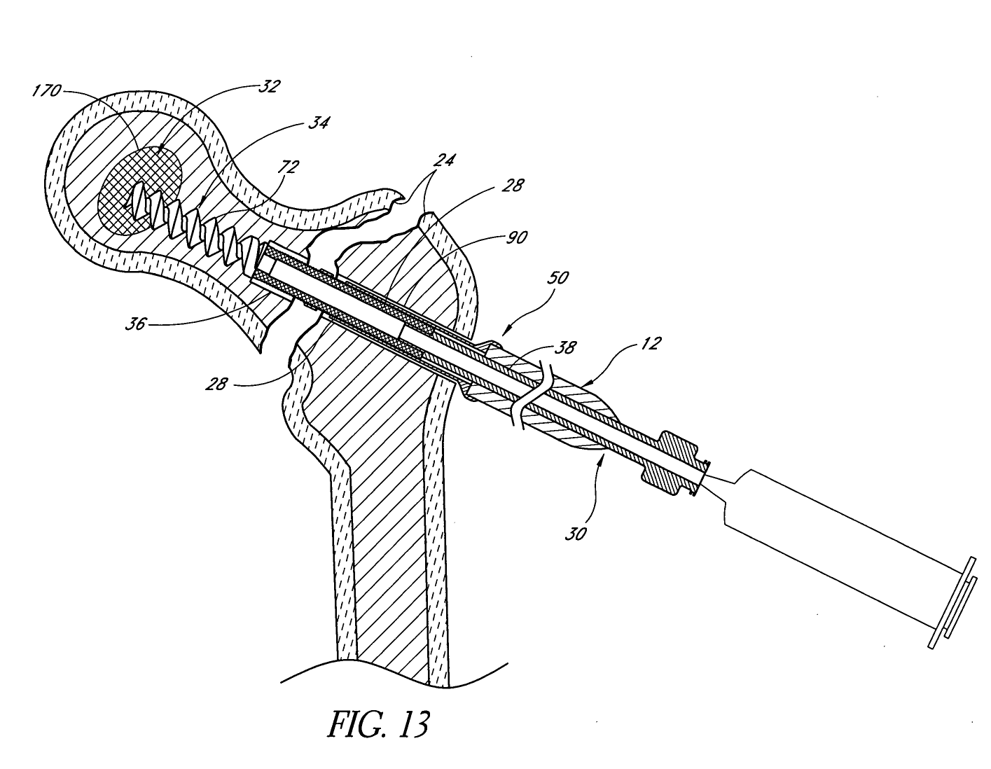

In one embodiment of operation, the guidewire 102 may be inserted into a bore 160 that is disposed through a bone, as shown in FIG. 9. For example, the bore 160 may extend through a portion of the femur 10. In the illustrated embodiment, the bore 160 extends through the gluteal tuberosity 22 and through the fracture 24 and into the head 14 of the femur 10. It is contemplated that the bore 160 can be formed through known drilling techniques. The fixation device 12 can then be slid over and along the guidewire 102 in the distal direction. In the illustrated embodiment, the guidewire 102 is disposed in the first and second lumens 43, 143 of the fixation device 12 and the portion of the lumen 109 extending through the handle 108. That is, the guidewire 102 be disposed through the first portion 36, the deliver pin 138, and the handle assembly 107. In other embodiments, it is contemplated that the fixation device 12 may or may not be used in combination with a guidewire. For example, the fixation device 12 may be delivered with the deployment device 100 without using a guidewire. As mentioned above, it is also anticipated that in certain embodiments the fixation device 12 is self-tapping and can be inserted across the fracture 24 without providing a bore 160.

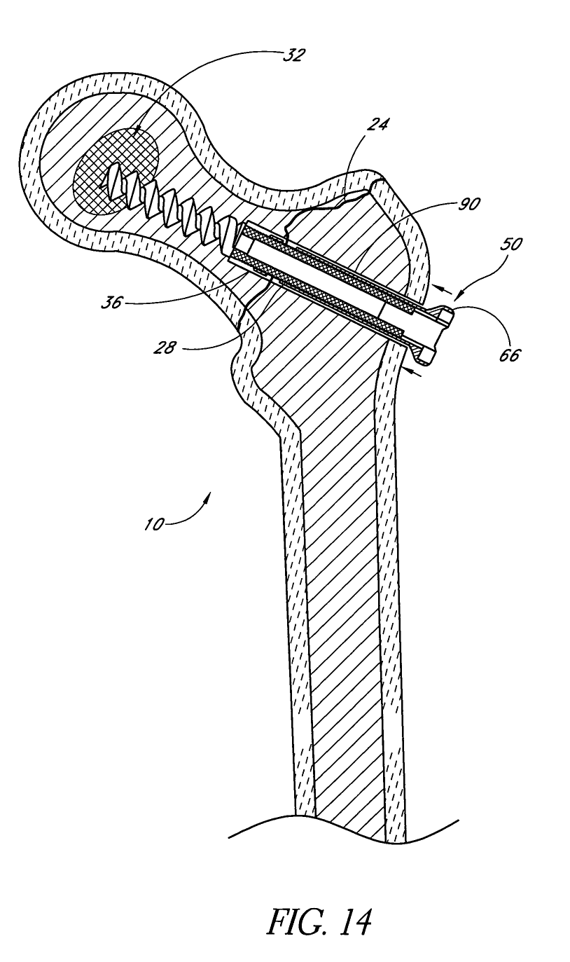

With the guidewire in place, the fixation device 12 may be advanced over the guidewire and into the bone. In the illustrated embodiment, the fixation device 12 is advanced by griping and rotating the handle 108 to rotate the fixation device 12. As the fixation device 12 is rotated, the distal bone anchor 34 is rotated and advanced through the femur 10 over the guidewire 102, as shown in FIG. 10. In one embodiment, the fixation device 12 is rotated and advanced in a distal direction until the distal bone anchor 34 is located within the head 14 of the femur 10. As illustrated in FIG. 11, once the fixation device 12 is in the desired location, the handle assembly 107 can be separated from the driver 106 to expose the proximal end of the pin 138 at the proximal end of the driver 106. Additionally, the guidewire 102 can be removed before or after the handle assembly 107 is separated from the driver 106. For example, the guidewire 102 can be proximally advanced out of the lumen 109 until the guidewire 102 is removed from the fixation device 12 and the hand assembly 107. After the guidewire 102 is remove from the delivery system 99, the delivery system 99 can deliver treatment material to the bone as disclosed below. However, it should be appreciated that in modified embodiments, the guidewire 102 may remain in the fixation device 12 thorough one or more of the delivery steps described below.

With respect to FIG. 12, a portion of the pin 138 is exposed so that a distal or tip 162 of a delivery device 164 (e.g., a syringe) can be inserted therein. At least a portion of the tip 162 can be inserted and advanced through the fitting 140 of the pin 138 providing access to the proximal end of the lumen 143. The syringe 164 can contain treatment material within its chamber 166. The clinician can move the plunger 168 in the distal direction to deliver material within the chamber 166 out through the tip 162 and into and through the handle assembly 107 and to the fixation device 12. The material can then pass through the pin 138 and the first portion 36. It should be appreciated any of a variety of mechanisms may be used as the delivery device 164. Such devices include, but are not limited to, compressible tubes, hand held trigger guns, and the like.

As shown in FIG. 13, the injection material 170 can surround at least a portion of the fixation device 12. In one embodiment, the material 170 is delivered out of the end of the helical locking structures 72. The interior surfaces of the helical locking structures 72 can define a passage or path that the material from the central lumen passes through and out of and into the bone. Thus, the material 170 is delivered out of the distal end of the helical locking structures 72 and may diffuse about the distal end of the fixation device 12 through the bone. Although not illustrated, the fixation device 12 can have fenestrations, holes or openings for delivering the material delivered by the syringe 164 out of the side of the fixation device 12.

In one embodiment, the guidewire 102 may be inserted back into the fixation device 12. This may advantageously push additional injection material 170 out of the lumens and into the bone. To facilitate such movement, the guidewire 102 may be provided with a plunging element (not shown) and/or a different guidewire with a plunging element may be used.

After the distal end of the fixation device 12 is in a desired position and desired amount of agent 170 has been deployed, the fracture 24 may be compressed in a variety of manners. In one embodiment, proximal retraction may be applied to the proximal end of the delivery pin 138 while a distal force is applied to the proximal anchor 50 through the driver 106. In this manner, the proximal anchor 50 and the body 28 can be compressed so that the proximal anchor 50 is advanced distally relative to the first portion 36 until the anchor 50 fits snugly against the outer surface of the femur or tissue adjacent to the femur and the fracture 24 is completely reduced as shown in FIG. 14. This may be done manually or with the aid of any of a variety of mechanisms configure to grip and apply proximal retraction to the delivery pin and/or a distal force to the driver 106. See e.g., U.S. application serial Ser. No. 10/790,670 filed Mar. 1, 2004, and U.S. application Ser. No. 10/790,671 filed Mar. 1, 2004, the disclosure of which are incorporated in their entirety herein by reference.

In another embodiment, the delivery system 99 including the driver 106 and/or the delivery pin 138 may be removed from the fixation device 12. Compression of the fracture may then be accomplished using any of the mechanisms described in the above referenced applications.

In yet another embodiment, after the delivery system 99 is removed, the clinician can apply a distal force on the flange 66 to distally advance the proximal anchor 50 and thereby reduce and close up the fracture 24. Advantageously, the proximal anchor 50 can be used to conveniently and gently manually close the fracture 24. In certain applications, the femur 10 may be weakened and have many voids due to osteoporosis or other conditions that may weaken the bone. When the proximal anchor 50 is manually advanced relative to the first portion 36, the force applied to the femur head 14 by the distal anchor 34 is reduced and may reduce the frequency of pull-out of the fixation device 12. That is, the delivery pin 138 may not be retracted to close the fracture 24.

In still another embodiment, the fracture may be closed, partially or wholly, before the fixation device is inserted across the fracture. After insertion, the proximal anchor 50 is distally advanced to reduce the length of the fixation device 12.

The fixation device 12 may be used to deliver medical agents to other portions of the bone and/or facture besides those portions in which the distal anchor resides. For example, the clinician can use known techniques to determine when the fixation device 12 reaches a first position. The medical agent may then be injected through the fixation device 12 as described above. Once the desired amount of agent is injected, the fixation device may be advanced further and the procedure completed as described above. In one embodiment, the first position is within or near the fracture 24. In this manner, a suitable medical agent (e.g., a growth factor) may be applied to the fracture 24 across which the fixation device extends. In another embodiment, the first position may correspond to the area between one or more joints and/or bones that are to be fused (e.g., adjacent vertebrae in the spine). In such an embodiment, the medical agent may comprise an agent that promotes bone fusion (e.g., a cement or graft material).

In another embodiment, the fixation device 12 is advanced distally along until the fixation device reaches a first position using known techniques to determine when the fixation device 12 reaches the first position. Then, the fixation device 12 is moved proximally or backed out a predetermined distance. In one embodiment, the fixation device 12 is backed out 10-15 mm from the first position. In another embodiment, the fixation device is backed out less than about 20 mm from the first position. In yet another embodiment, the fixation device is backed out less than about 15 mm from the first position. In another embodiment, the fixation device is backed out more than about 5 mm from the first position. In some embodiments, the clinician can rotate the handle 107 to cause the fixation device 12 to move in the proximal direction a predetermined amount. The guidewire 102 may or may not be removed before the fixation device 12 is backed out.

After the fixation device 12 has been backed out the desired distance, the medical agent may be injected through the fixation device as described above. In one embodiment, this involves removing the handle 108 from the driver 106 to expose the fitting 140. The agent 170 can fill the space previously occupied by the distal end 32 of the fixation device 12 before it was backed out. After the desired amount of material has been deployed out of the fixation device 12, the fixation device may be advanced in the distal direction until the distal anchor reaches the desired location. In one embodiment, this involves reattaching the handle 108 to the driver 106. The fixation device may thereafter be compresses as described above. To detach the pin 138 from the first portion 36, the delivery pin 138 can be rotated relative to the first portion 36. The clinician can conveniently grip the fitting 140 to rotate the pin 138.

The above-described procedure is particularly advantageous for treating osteoportic or otherwise weakened bone, especially with respect to hip fractures. Injectible medical agents (e.g., bone cements) may optimize the stability of hip fractures by augmenting structural bone deficits in areas of compromised cancellous bone, such as in the proximal femur. The above-described procedure allows the agent to be injected into the cancellous bone and then the distal anchor may then be advanced into the filled area. This creates a tight bond between the injected material and the distal anchor increasing the internal fixation of the device.

The above-described methods and apparatuses provide several advantages for delivering a medical agent into a bone. As shown in FIG. 8B, the junction between the delivery pin 138 and the first portion 36 of the body is advantageously positioned within the housing 52 of the proximal anchor 50. In this manner, the housing 52 helps to prevent leakage of medical agent though the junction. In addition, the configuration of the junction itself may limit leakage. For example, a threaded connection between the first portion 36 and the delivery pin 138 is a particularly effective connection for reducing leakage. Another advantage is that the proximal end 142 of the delivery pin 138 be positioned near the handle assembly 107 outside of the patient. In this manner, the junction between the delivery pin 138 and the delivery device (e.g., syringe) is positioned such that any leakage therebetween occurs outside of the patient. Another advantage is that the delivery pin 138 and body 36 provide a large cross-sectional area through which to the agent can be injected. Many medical agents are quite viscous and thus a large amount of force may be required to inject the agent though the fixation device 12. Accordingly, it may not be feasible to insert a second tubular member into the lumen of the fixation device to inject the agent.

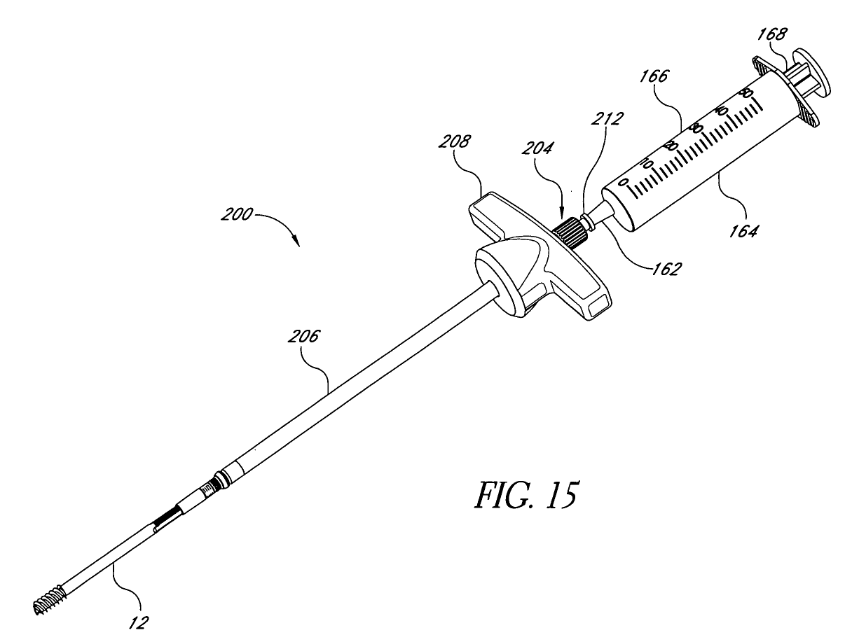

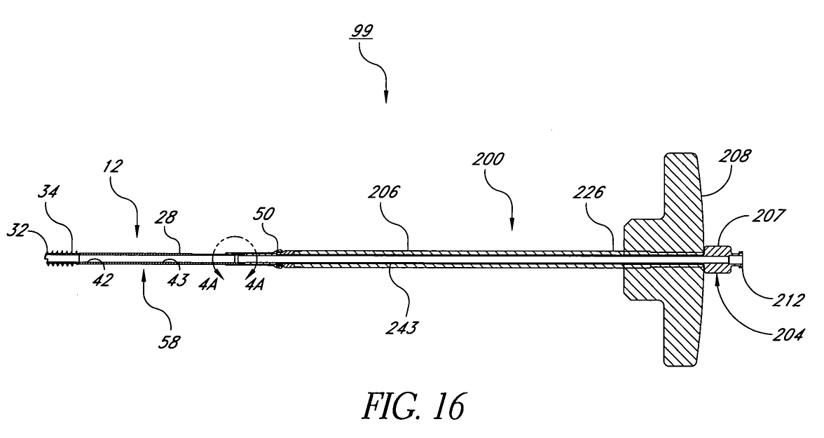

FIGS. 15 and 16 illustrate a modified embodiment of a deployment system 200. In the illustrated embodiment, the deployment system 200 includes a delivery pin 204, a handle 208, and a driver 206. In this embodiment, the driver 206 and handle 208 are generally similar to the driver 106 and handle 107 described above. However, in this embodiment, the driver 206 and handle 208 may be integrally or otherwised coupled together. Similarly, the delivery pin 204 is similar to the delivery pin described above. Accordingly, the distal end of the pin 204 is configured to releasably engage the first portion 36 of the fixation device 12. The delivery pin 204 is configured to extend through the lumen of the driver 206 and the handle 208 such that the proximal end of the delivery pin 204 is positioned on a proximal side of the handle 208. A coupling or fitting member 212 (e.g., a luer connector) is preferably provided on the proximal end of the pin 204. The proximal end of the pin 204 may also include a flange 207 for rotating the pin with respect to the driver 206 and handle 208. The coupling 212 is configured to receive the tip 162 of the syringe 164 or another type of delivery device for the medical agent.

As shown in FIG. 15, the delivery device (e.g., syringe) 164 can deliver material to the proximal end of the pin 204. When the tip 162 of the syringe 164 is engaged with the pin 204, material is delivered by the syringe 164 into the delivery pin 204 and into the lumen 243 of the pin 204. The material passes through the lumen 243 and out of the distal end of the pin 204. In this manner, material feed from the syringe 164 can pass through the delivery system 200 and into the bone. After a desired amount of material has been delivered, the clinician can grip and rotate the knob 207 of the proximal end of the pin 204 to detach the pin 204 from the distal anchor 34. The delivery pin 204 can then be drawn distally out of the driver 206 and the handle 208.

As described above, the guidewire may be removed or remain in the fixation device 12 during the delivery steps. In addition, the guidewire or an additional element may be used as a plunger to push additional treatment material through the lumens.

The delivery device 200 may also be used insert the fixation device 12 and/or compress a fracture. For example, rotation of the handle 208 causes the fixation device 12 to rotate. Proximal retraction can be achieved by pulling on the proximal end of the delivery pin 204 while holding the handle 208 stationary and/or pushing the handle 208 in a distal direction. The delivery pin 204 may be separated from the fixation device 12 by rotating the delivery pin 204 to decouple the pin from the first portion 306 of the body. As with the embodiments described above, the delivery device may be used in combination with a separate insertion mechanisms such as the mechanism described in U.S. application Ser. No. 10/790,670 filed Mar. 1, 2004, and U.S. application Ser. No. 10/790,671 filed Mar. 1, 2004, the disclosure of which are incorporated in their entirety herein by reference.

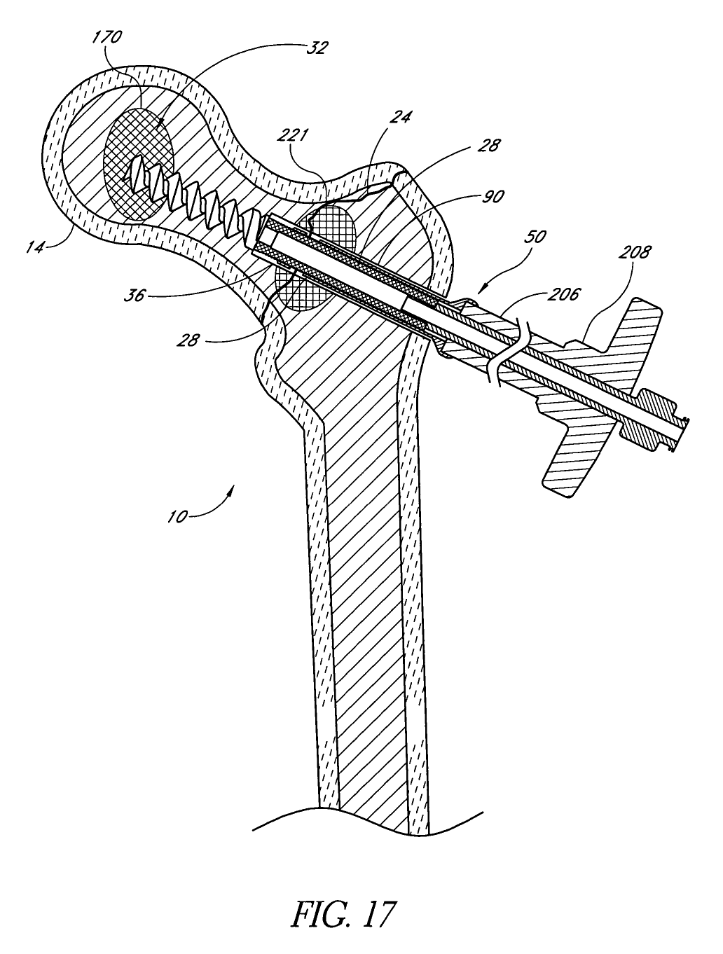

With respect to FIG. 17, the delivery device 200 may be used to delivery a medical agent to the portions of the bone occupied by the distal anchor, the facture and/or other portions of the bone. For example, when the distal anchor 34 of the fixation device 12 reaches a desired position generally corresponding to the fracture 24, a syringe 164 filled with a medical agent (e.g., growth factor) can be used to deliver the agent through the delivery system 99. Thus, the agent can then be delivered out of the fixation device 12 and into the portion of the bone surrounding the fracture 24. After a desired amount of agent has been delivered, the syringe 164 can be removed from the handle 208. The fixation device 12 can then be distally advanced by rotating the handle 208 until the fixation device 12 reaches a second desired location within the femur 10. After the fixation device 12 reaches the second desired location, a second syringe 164 filled with the same or different agent (e.g., bone cement) can then deliver agent through and out of the delivery system 200 which then deploys the agent into the femoral head 14. In a modified embodiment, the agent is injected after the fixation device 12 has been distally advanced to a first location and then can be backed out about a predetermined amount as described above. Although not illustrated, a guidewire can be used to access the femur 10. However, the guidewire is preferably removed from the delivery system 200 before the treatment agent is delivered through the delivery system 200 and into the bone.

In the above described method, the first agent may be a growth factor comprising growth factor agent and carrier material. For example, the syringe 164 may deliver growth factor which comprises a carrier (e.g., collagen polymer, or the like) to deliver the growth factor. The growth factor may be one of a bone metamorphic protein, fibroblast growth factors, platelet-derived growth factor, TGF-beta, and/or other suitable proteins for causing regeneration of the bone. It is contemplated that one of ordinary skill in the art can determine the-appropriate combination and amount of growth factors in combination with the carrier material to achieve the desired regeneration of the bone.

The second agent may be a bone cement configured to improve the structural properties of the femur 10. In one embodiment, the bone cement is biodegradable bone cement that can provide mechanical reinforcement at the fracture site for promoting fracture healing. The biodegradable bone cement can then be resorbed over a period of time, preferably when the healing bone will provide increased structural support. However, in other embodiments, non-biodegradable bone cement can be used with the fixation device 12.

It should be appreciated that medical agents described above are exemplary. As such, even though the medical agents mentioned have specific advantages, the above described apparatuses and methods should not be limited to the specific medical agents or type of medical agents described above.

It is contemplated that one or more of the above mentioned procedures can be perfOormed to deploy one or more fixation devices 12 in the bone. Optionally, each fixation device 12 can be used to deploy treatment material to the bone. Thus, a plurality of bone fixation devices can be delivered into the bone, and each fixation device may be used to deliver treatment material to one or more locations in the bone.

The fixation devices 12 of the present invention may be made from either conventional bioabsorbable materials or conventional non-absorbable materials, combinations thereof and equivalents thereof. In addition, natural materials such as allografts may be used. Examples of absorbable materials include homopolymers and copolymers of lactide, glycolide, trimethylene carbonate, caprolactone, and p-dioxanone and blends thereof. The following two blends may be useful: 1) the blend of poly(p-dioxanone) and a lactide/glycolide copolymer, as disclosed in U.S. Pat. No. 4,646,741 which is incorporated by reference and (2) the glycolide-rich blend of two or more polymers, one polymer being a high lactide content polymer, and the other being a high glycolide content disclosed in U.S. Pat. No. 4,889,119 which is incorporated by reference. Additional bioabsorbable materials are disclosed in copending application Ser. No. 09/558,057 filed Apr. 26, 2000, the disclosure of which is incorporated in its entirety herein by reference.

The fixation devices 12 may also be made from conventional non-absorbable, biocompatible materials including stainless steel, titanium, alloys thereof, polymers, composites and the like and equivalents thereof. In one embodiment, the distal anchor (e.g., distal anchor 34) comprises a metal helix, while the body and the proximal anchor (e.g., proximal anchor 50) comprise a bioabsorbable material. Alternatively, the distal anchor 34 comprises a bioabsorbable material, and the body and proximal anchor 50 comprise either a bioabsorbable material or a non-absorbable material. As a further alternative, each of the distal anchor 34 and the body may comprise a non-absorbable material, connected by an absorbable link. This may be accomplished by providing a concentric fit between the distal anchor and the body, with a transverse absorbable pin extending therethrough. This embodiment will enable removal of the body following dissipation of the pin, while leaving the distal anchor 34 within the bone.

The components of the invention (or a bioabsorbable polymeric coating layer on part or all of the anchor surface), may contain one or more bioactive substances, such as antibiotics, chemotherapeutic substances, angiogenic growth factors, substances for accelerating the healing of the wound, growth hormones, antithrombogenic agents, bone growth accelerators or agents, and the like. Such bioactive implants may be desirable because they contribute to the healing of the injury in addition to providing mechanical support.

In addition, the components may be provided with any of a variety of structural modifications to accomplish various objectives, such as osteoincorporation, or more rapid or uniform absorption into the body. For example, osteoincorporation may be enhanced by providing a micropitted or otherwise textured surface on the components. Alternatively, capillary pathways may be provided throughout the body and collar, such as by manufacturing the anchor and body from an open cell foam material, which produces tortuous pathways through the device. This construction increases the surface area of the device which is exposed to body fluids, thereby generally increasing the absorption rate. Capillary pathways may alternatively be provided by laser drilling or other technique, which will be understood by those of skill in the art in view of the disclosure herein. In general, the extent to which the anchor can be permeated by capillary pathways or open cell foam passageways may be determined by balancing the desired structural integrity of the device with the desired reabsorption time, taking into account the particular strength and absorption characteristics of the desired polymer.

One open cell bioabsorbable material is described in U.S. Pat. No. 6,005,161 as a poly(hydroxy) acid in the form of an interconnecting, open-cell meshwork which duplicates the architecture of human cancellous bone from the iliac crest and possesses physical property (strength) values in excess of those demonstrated by human (mammalian) iliac crest cancellous bone. The gross structure is said to maintain physical property values at least equal to those of human, iliac crest, cancellous bone for a minimum of 90 days following implantation. The disclosure of U.S. Pat. No. 6,005,161 is incorporated by reference in its entirety herein.

The components of the present invention may be sterilized by any of the well known sterilization techniques, depending on the type of material. Suitable sterilization techniques include heat sterilization, radiation sterilization, such as cobalt 60 irradiation or electron beams, ethylene oxide sterilization, and the like.

The specific dimensions of any of the bone fixation devices of the present invention can be readily varied depending upon the intended application, as will be apparent to those of skill in the art in view of the disclosure herein. Moreover, although the present invention has been described in terms of certain preferred embodiments, other embodiments of the invention including variations in dimensions, configuration and materials will be apparent to those of skill in the art in view of the disclosure herein. In addition, all features discussed in connection with any one embodiment herein can be readily adapted for use in other embodiments herein. The use of different terms or reference numerals for similar features in different embodiments does not imply differences other than those which may be expressly set forth. Accordingly, the present invention is intended to be described solely by reference to the appended claims, and not limited to the preferred embodiments disclosed herein.

We claim:

1. A method of securing a first bone fragment to a second bone fragment, comprising:
providing a fixation device comprising an elongate body and a proximal anchor, the elongate body having a proximal end, a distal end with a distal anchor thereon and a lumen extending through the elongate body, the proximal anchor being carried by the elongate body and having complementary structures for permitting axial travel of the proximal anchor with respect to the elongate body in a distal direction but resisting axial travel of the proximal anchor with respect to the elongate body in a proximal direction;
providing a delivery pin having a distal end, a proximal end and delivery lumen extending therethrough, the distal end of the delivery pin being configured to engage the proximal end of the elongate body;
distally advancing the fixation device to a first position with respect to the first and second bone fragments;
delivering a medical agent to the fixation device by injecting the medical agent through the delivery lumen of the delivery pin and into the lumen of the elongate body; and
detaching the delivery pin from the elongate body.

2. The method of claim 1, further comprising coupling the delivery pin to the elongate body before distally advancing the fixation device to the first position.

3. The method of claim 2, wherein distally advancing the fixation device to the first position comprises rotating the fixation device.

4. The method of claim 1, further comprising:
proximally retracting the fixation device from a second position to the first position with respect to the first and second bone fragments; and
distally advancing the fixation device from the first position.

5. The method of claim 4, wherein the medical agent comprises a bone cement.

6. The method of claim 1, further comprising:
providing a delivery device including a tubular driver and a handle configured for rotating the driver, the driver having a distal end removably coupled to the proximal anchor and configured for rotating the proximal anchor, the delivery pin extending through a central lumen of the driver.

7. The method of claim 6, further comprising removing the handle from the tubular driver to expose the proximal end of the delivery pin extending from the driver.

8. The method of claim 1, wherein the medical agent comprises an orthobiologic agent.

9. The method of claim 8, wherein the orthobiologic agent comprises one of growth factor, bone cement, and combinations thereof.

10. The method of claim 1, further comprising inserting a tip of a syringe into the proximal end of the delivery pin and compressing the syringe to inject the medical agent into the delivery pin.

11. The method of claim 1, further comprising advancing the fixation device over a guidewire.

12. The method of claim 11, further comprising removing the guidewire from the fixation device before delivering the medical agent to the first position.

13. The method of claim 12, further comprising advancing a plunger through the delivery pin and elongate body.

14. The method of claim 13, wherein the plunger comprise the guidewire.

15. A bone fixation system comprising:
a fixation device comprising an elongate body and a proximal anchor, the elongate body having a proximal end, a distal end with a distal anchor thereon and a lumen extending through the elongate body, the proximal anchor being carried by the elongate body and having complementary structures for permitting axial travel of the proximal anchor with respect to the elongate body in a distal direction but resisting axial travel of the proximal anchor with respect to the elongate body in a proximal direction;
a delivery pin having a distal end, a proximal end and a delivery lumen extending therethrough, the distal end of the delivery pin configured to be coupled to the fixation device to place the delivery lumen in communication with the first lumen;
an elongated drive body having a proximal end and a distal end, a cannula extending through the elongated drive body and configured to surround a portion of the delivery pin when the distal end of the elongated drive body is coupled to the proximal anchor of the fixation device; and
a handle releasable coupled to the proximal end of the elongated driver body;
wherein removing the handle from the elongated drive body exposes the proximal end of the delivery pin which extends through the elongated drive body.

16. The bone fixation system as in claim 15, further comprising a delivery system comprising a delivery agent, the delivery system configured to mate with the proximal end of the delivery pin.

17. The bone fixation system as in claim 16, wherein the delivery system comprises a syringe.

18. The bone fixation system as in claim 15, wherein the distal anchor comprise a helical flange.

19. The bone fixation system as in claim 15, further comprising an anti-rotational lock between the elongate body and the proximal anchor includes an anti-rotational lock.

20. A bone fixation system comprising:
a fixation device comprising an elongate body and a proximal anchor, the elongate body having a proximal end, a distal end with a distal anchor thereon and a lumen extending through the elongate body, the proximal anchor being carried by the elongate body and having complementary structures for permitting axial travel of the proximal anchor with respect to the elongate body in a distal direction but resisting axial travel of the proximal anchor with respect to the elongate body in a proximal direction;
an elongated drive body having a proximal end, a distal end and a cannula extending therethrough, the distal end of the elongated drive body configured to engage the proximal anchor such that rotation of the drive body causes rotation of the proximal anchor; the proximal end of the drive body including a handle; and
a delivery pin having a distal end, a proximal end and a delivery lumen extending therethrough, the distal end of the delivery pin configured to be coupled to the fixation device to place the delivery lumen in communication with the first lumen, the delivery pin extending through the cannula of the elongated body such that the distal end of the delivery pin may be coupled to the proximal end of the elongate body while the proximal end of the elongate body extends out through the handle.

21. The bone fixation system as in claim 20, further comprising a delivery system comprising a delivery agent, the delivery system configured to mate with the proximal end of the delivery pin.

22. The bone fixation system as in claim 21, wherein the delivery system comprises a syringe.

23. The bone fixation system as in claim 20, wherein the distal anchor comprises a helical flange.

24. The bone fixation system as in claim 20, comprising an anti-rotational lock between the elongate body and the proximal anchor.

25. A method of securing a first bone fragment to a second bone fragment, comprising:
distally moving a fixation device to a first position, the fixation device comprising an elongate body, having a proximal end and a distal end, a helical anchor on the distal end of the elongate body and a proximal anchor moveably carried by the elongate body, the elongate body and the proximal anchor having complementary retention structures configured to resist proximal movement of the proximal anchor with respect to the elongate body;
delivering a first material through and out of the fixation device when the fixation device is located in the first position;
moving the fixation device from the first position to a second position, the second position being distal to the first position; and
delivering a second material through and out of the fixation device when the fixation device is in the second position.

26. The method of claim 25, further comprising:
distally advancing the fixation device from the first position to a third position after the first material has been delivered out of the fixation device;
proximally moving the fixation device from the third position to the second position; and
distally moving the fixation device after the second material is delivered from the fixation device.

27. The method of claim 25, wherein the first material comprises a growth factor and the second material comprises bone cement.

28. A femoral neck fracture fixation device, comprising:
an elongate body comprising a distal portion, a delivery pin and lumen extending therethrough, the distal portion including a helical bone anchor, the delivery pin and distal portion being detachably coupled to each other at a junction;
a proximal anchor, moveably carried by the elongated body and comprising a tubular sleeve that in a first position extends distally past the junction between the distal portion and the delivery pin;
complementary retention structures between the proximal anchor and the elongate body, the complementary retention structures configured to restrain proximal movement of the proximal anchor with respect to the elongate.body; and
a fitting coupled to a proximal end of the delivery pin, the fitting configured to receive a tip of a medical agent delivery device.

29. The fixation device of claim 28, wherein the medical agent delivery device comprises a syringe.
